(19) United States
(12) Patent Application Publication
(10) Pub. No.: US 2025/0316332 A1
(43) Pub. Date: Oct. 9, 2025

(54) METHODS AND SYSTEM FOR USING METHYLATION DATA FOR DISEASE DETECTION AND QUANTIFICATION

(71) Applicant: Personalis, Inc., Fremont, CA (US)

(72) Inventors: John LYLE, Fremont, CA (US); Qi ZHANG, Fremont, CA (US)

(21) Appl. No.: 18/866,825

(22) PCT Filed: May 19, 2023
(86) PCT No.: PCT/US2023/067253

§ 371 Date: Nov. 18, 2024

Related U.S. Application Data
(60) Provisional application No. 63/343,878, filed on May 19, 2022.

(51) Int. Cl. G16B 20/20; C12Q 1/6869; C12Q 1/6886; G16B 30/00; G16B 50/30

(52) U.S. Cl. CPC G16B 20/20; C12Q 1/6869; C12Q 1/6886; G16B 30/00; G16B 50/30

(57) ABSTRACT

Provided herein are methods and system for using methylation data to improve disease detection.

### CROSS REFERENCE TO RELATED APPLICATIONS

This application claims priority to U.S. Provisional Application No. 63/343,878, filed May 19, 2022, which is entirely incorporated herein by reference.

### BACKGROUND

Detecting and monitoring cancer is complicated by the fact that sequencing errors and statistical noise can be of such magnitude to obscure signals that are needed to detect cancer and/or to detect meaningful changes. This can lead to delays in diagnoses, delays in treatments, delays to changing from ineffective treatment, etc. Thus, there is a need to improve the sensitivity and specificity of disease.

### BRIEF SUMMARY

In one aspect, the present disclosure provides a method comprising: (a) accessing sequencing data that had been generated by sequencing a processed sample obtained from a subject, the sequencing data including or having been based on a set of sequence reads; (b) identifying, using the sequencing data, one or more loci corresponding to single nucleotide polymorphisms (SNPs) at which at least a threshold number or percentage of the set of sequence reads included a base identifier that departed from a reference base identifier corresponding to a same position in a reference sequence; (c) for each locus of the one or more loci: (i) determining, for each of one or more positions within a sequence portion that includes the locus, a methylation percentage using reads that include the corresponding SNP, and (ii) identifying, for each of the one or more positions corresponding to the sequence portion that includes the locus, a comparative methylation percentage; (d) generating a result based on each determined methylation percentage and each comparative methylation percentage, wherein the result represents a prediction as to whether the sample is associated with a particular medical condition, whether the sample is associated with a medical condition of a particular stage, whether the subject has a particular type of medical condition, or whether the sample was collected from a specific individual; and (e) outputting the result.

In a further embodiment and in accordance with the above, generating the result includes performing a statistical analysis that indicates, for at least one locus of the one or more loci, a probability of sequencing error accounting for a subset of reads that include the SNP also having the methylation percentage.

In a further embodiment and in accordance with any of the above, for each locus of the one or more loci, the comparative methylation percentage is identified using a look-up technique that uses the reference sequence or another reference sequence.

In a further embodiment and in accordance with the above, (i) the one or more loci comprises a plurality of loci; (ii) the comparative methylation percentage for a first subset of the plurality of loci is identified using a look-up technique that uses at least part of population-level sequencing data as a first reference sequence; and (iii) the comparative methylation percentage for a second subset of the plurality of loci is identified using a look-up technique that uses at least part of subject-specific normal sequencing data as a second reference sequence.

In a further embodiment and in accordance with the above, the population-level sequencing data is based on or extracted from one or more databases.

In a further embodiment and in accordance with the above, the one or more databases comprises one or more methylation databases or one or more polymorphism databases.

In a further embodiment and in accordance with the above, the one or more databases comprises one or more publicly available databases or one or more proprietary databases.

In a further embodiment and in accordance with any of the above, further comprising, for each locus of the one or more loci: (i) defining a first subset of reads aligned to at least part of the sequence portion to include reads that include the SNP; (ii) defining a second subset of reads aligned to at least part of the sequence portion to include reads that do not include the SNP and instead include the reference base identifier; and (iii) generating, for each position of the one or more positions, the comparative methylation percentage using the methylation state of each cytosine aligned to the position in the second subset of reads.

In a further embodiment and in accordance with any of the above, further comprising, for a particular locus of the one or more loci: (i) detecting, using the sequencing data, one or more CpG sites that are within a predefined number of positions from the SNP; and (ii) defining the one or more positions to be the loci of the cytosine nucleotide within each of the one or more CpG sites.

In a further embodiment and in accordance with any of the above, (i) the sample was a blood sample; (ii) the result represents a prediction that the sample is associated with the particular condition; and (iii) the particular condition includes cancer.

In a further embodiment and in accordance with the above, levels of circulating tumor DNA were below 5 parts per million in the blood sample.

In a further embodiment and in accordance with any of the above, the accessed sequencing data was enriched using a plurality of capture probes.

In a further embodiment and in accordance with the above, the plurality of capture probes comprises one or more self-identifying capture probes.

In a further embodiment and in accordance with the above, the plurality of capture probes comprises 1200 or more capture probes.

In a further embodiment and in accordance with the above, the plurality of capture probes comprises 1800 or more capture probes.

In another aspect, the present disclosure provides a method comprising: (a) accessing solid-tumor sequencing data that had been generated by sequencing a processed sample of a solid tumor obtained from a subject, the sequencing data including or having been based on a set of sequence reads; (b) determining, for each position of a set of positions in a genome: (i) a solid-tumor-sample-specific methylation percentage that indicates a first proportion of bases in the solid-tumor sequencing data set that were aligned to the position and were methylated, and (ii) a comparative methylation percentage that indicates a second proportion of bases in a population sequencing data set or a subject-specific normal sequencing data set, or a combination thereof, that were aligned to the position and were methylated; (c) determining a subset of the set of positions for which the solid-tumor-sample-specific methylation percentage was sufficiently different from the comparative methylation percentage; (d) accessing cell-free sequencing data that had been generated by sequencing cell free DNA in a processed or unprocessed sample of the subject; (e) detecting, for each position of the subset of the set of positions, a quantity of bases aligned to the position that were methylated; and (f) outputting a result based on, for each position of the subset, the quantity of bases aligned to the position that were methylated.

In a further embodiment and in accordance with the above, for each position of the set of positions in the genome: (i) at least a first portion of the comparative methylation percentage that indicates a first proportion of bases is identified using a look-up technique that uses at least part of population-level sequencing data as a first reference sequence; and (ii) at least a second portion of the comparative methylation percentage that indicates a second proportion of bases is identified using a look-up technique that uses at least part of subject-specific normal sequencing data as a second reference sequence.

In a further embodiment and in accordance with the above, the population-level sequencing data is based on or extracted from one or more databases.

In a further embodiment and in accordance with the above, the one or more databases comprises one or more methylation databases or one or more polymorphism databases.

In a further embodiment and in accordance with the above, the one or more databases comprises one or more publicly available databases or one or more proprietary databases.

In a further embodiment and in accordance with any of the above, the method further comprises: (i) detecting one or more SNPs within the solid-tumor sequencing data set; (ii) detecting, using the solid-tumor sequencing data and for each of the one or more SNPs, one or more CpG sites that are within a predefined number of positions from the SNP; and (iii) defining the set of positions to be the loci of the cytosine nucleotide within each of the one or more CpG sites.

In a further embodiment and in accordance with any of the above, the method further comprises: (i) using the solid-tumor sequencing data to detect one or more SNPs; and (ii) detecting, for each SNP of the one or more SNPs, which of a second set of sequence reads include the SNP, wherein the cell-free sequencing data includes the second set of sequence reads, and wherein the result is further based on a quantity of reads in the second set of sequence reads for which it was detected that the read included the SNP.

In a further embodiment and in accordance with any of the above, the method further comprises generating an estimated prevalence of circulating tumor DNA to circulating non-tumor DNA based on the quantity, for each of the subset of the set of positions, of the bases aligned to the position that were methylated, wherein the result includes the estimated prevalence.

In a further embodiment and in accordance with any of the above, the result includes a level of circulating tumor DNA generated based on the quantity, for each of the subset of the set of positions, of the bases aligned to the position that were methylated.

In a further embodiment and in accordance with any of the above, levels of circulating tumor DNA were below 5 parts per million in the processed or unprocessed sample.

In a further embodiment and in accordance with any of the above, the method further comprises estimating a degree to which a disease of the subject has progressed or a probability that a disease of the subject is in remission based on the result.

In a further embodiment and in accordance with any of the above, the accessed sequencing data was enriched using a plurality of capture probes.

In a further embodiment and in accordance with the above, the plurality of capture probes comprises one or more self-identifying capture probes.

In a further embodiment and in accordance with the above, the plurality of capture probes comprises 1200 or more capture probes.

In a further embodiment and in accordance with the above, the plurality of capture probes comprises 1800 or more capture probes.

In another aspect, the present disclosure provides a method comprising: (a) accessing sequencing data that had been generated by sequencing a processed sample obtained from a subject, the sequencing data including or having been based on a set of sequence reads; (b) identifying, using the sequencing data, a plurality of loci corresponding to single nucleotide polymorphisms (SNPs) at which at least a threshold number or percentage of the set of sequence reads included a base identifier that departed from a reference base identifier corresponding to a same position in a reference sequence; (c) for each locus of the plurality of loci: (i) determining, for each of one or more positions within a sequence portion that includes the locus, a methylation percentage using reads that include the corresponding SNP, and (ii) identifying, for each of the one or more positions corresponding to the sequence portion that includes the locus, a comparative methylation percentage, wherein: (1) a first subset of the plurality of loci is identified using a look-up technique that uses at least part of population-level sequencing data as a first reference sequence, and (2) a second subset of the plurality of loci is identified using a look-up technique that uses at least part of subject-specific normal sequencing data as a second reference sequence; (d) generating a result based on each determined methylation percentage and each comparative methylation percentage, wherein the result represents a prediction as to whether the sample is associated with a particular medical condition, whether the sample is associated with a medical condition of a particular stage, whether the subject has a particular type of medical condition, or whether the sample was collected from a specific individual; and (e) outputting the result.

In a further embodiment and in accordance with the above, the population-level

sequencing data is based on or extracted from one or more databases.

In a further embodiment and in accordance with the above, the one or more databases comprises one or more methylation databases or one or more polymorphism databases.

In a further embodiment and in accordance with the above, the one or more databases comprises one or more publicly available databases or one or more proprietary databases.

In a further embodiment and in accordance with any of the above, the accessed sequencing data was enriched using a plurality of capture probes.

In a further embodiment and in accordance with the above, the plurality of capture probes comprises one or more self-identifying capture probes.

In a further embodiment and in accordance with the above, the plurality of capture probes comprises 1200 or more capture probes.

In a further embodiment and in accordance with the above, the plurality of capture probes comprises 1800 or more capture probes.

In a further embodiment and in accordance with any of the above, generating the result includes performing a statistical analysis that indicates, for at least one locus of the plurality of loci, a probability of sequencing error accounting for a subset of reads that include the SNP also having the methylation percentage.

In a further embodiment and in accordance with any of the above, the method further comprises, for each locus of the plurality of loci: (i) defining a first subset of reads aligned to at least part of the sequence portion to include reads that include the SNP; (ii) defining a second subset of reads aligned to at least part of the sequence portion to include reads that do not include the SNP and instead include the reference base identifier; and (iii) generating, for each position of the one or more positions, the comparative methylation percentage using the methylation state of each cytosine aligned to the position in the second subset of reads.

In a further embodiment and in accordance with any of the above, the method further comprises, for a particular locus of the plurality of loci: (i) detecting, using the sequencing data, one or more CpG sites that are within a predefined number of positions from the SNP; and (ii) defining the one or more positions to be the loci of the cytosine nucleotide within each of the one or more CpG sites.

In a further embodiment and in accordance with any of the above, (i) the sample was a blood sample; (ii) the result represents a prediction that the sample is associated with the particular condition; and (iii) the particular condition includes cancer.

In a further embodiment and in accordance with the above, levels of circulating tumor DNA were below 5 parts per million in the blood sample.

In another aspect, the present disclosure provides a method comprising: (a) accessing sequencing data of a biological sample of a subject, wherein the biological sample: (i) included a plurality of nucleic acid molecules, (ii) was enriched using a self-identifying capture probe of a probe set, and (iii) was enriched for a first set of nucleic acid molecules of the plurality of nucleic acid molecules, wherein each nucleic acid molecule of the first set of nucleic acid molecules includes a first target sequence; (b) determining, based on the sequencing data, a first amount of the first set of nucleic acid molecules; (c) identifying a probe-set identifier of the probe set based on the first amount of the first set of nucleic acid molecules; (d) generating, based on the probe-set identifier, a result indicating that the probe set includes one or more subject-specific capture probes that enrich the biological sample for a second set of nucleic acid molecules of the plurality of nucleic acid molecules, wherein the second set of nucleic acid molecules facilitate a determination of a classification of pathology for the subject; and (e) outputting the result.

In a further embodiment and in accordance with the above, determining the first amount of the first set of nucleic acid molecules includes: (i) sequencing the plurality of nucleic acid molecules of the enriched biological sample to obtain a plurality of sequence reads; (ii) aligning each of the plurality of sequence reads to a corresponding portion of a human reference genome; (iii) identifying, from the aligned sequence reads, a set of sequence reads that correspond to the first target sequence; (iv) determining an amount of the set of sequence reads; and (v) identifying, based on the amount of the set of sequence reads, a sequencing coverage for the probe set.

In a further embodiment and in accordance with the above, identifying the sequencing coverage for the probe set includes: (i) determining a distribution of the aligned sequence reads across a genomic region that corresponds to the first sequence: (ii) identifying a peak within the distribution, wherein the peak indicates a particular location of the genomic region to which a largest amount of sequence reads are aligned; (iii) determining, based on the identified peak, a metric that represents the sequencing coverage; and (iv) identifying the probe-set identifier using the metric.

In a further embodiment and in accordance with any of the above, the method further comprises: (i) determining that the sequencing coverage exceeds a predetermined threshold; and (ii) in response to determining that the sequencing coverage exceeds the predetermined threshold, determining a first value of the probe-set identifier, wherein the first value is predictive of a presence of the first target sequence in the biological sample.

In a further embodiment and in accordance with any of the above, the method further comprises: (i) determining that the sequencing coverage does not exceed a predetermined threshold; and (ii) in response to determining that the sequencing coverage does not exceed the predetermined threshold, determining a second value of the probe-set identifier, wherein the second value is predictive of an absence of the first target sequence in the biological sample.

In a further embodiment and in accordance with any of the above, the first target sequence corresponds to a particular portion of the human reference genome.

In a further embodiment and in accordance with any of the above, the probe set further includes a normalizing capture probe, the method further comprising: (i) applying, to the biological sample, the normalizing capture probe to enrich the biological sample for a third set of nucleic acid molecules of the plurality of nucleic acid molecules, wherein each nucleic acid molecule of the third set of nucleic acid molecules includes a second target sequence; (ii) determining a second amount of the third set of nucleic acid molecules; (iii) determining a statistical value based on the second amount; and (iv) identifying the probe-set identifier based on the statistical value.

In another aspect, the present disclosure provides a method comprising: (a) accessing sequencing data of a biological sample of a subject, wherein the sequencing data includes a plurality of sequence reads, wherein each of the plurality of sequence reads align to a corresponding portion of a reference sequence, and wherein the biological sample: (i) included a plurality of nucleic acid molecules, (ii) was enriched using a self-identifying capture probe of a probe set, and (iii) was enriched for a first set of nucleic acid molecules of the plurality of nucleic acid molecules, wherein each nucleic acid molecule of the first set of nucleic acid molecules includes a first target sequence; (b) analyzing the sequencing data to identify a probe-set identifier of the probe set, wherein the analysis includes, for each region of the set of regions of the reference sequence: (i) determining an amount of sequence reads that align to the region, and (ii) comparing the amount of sequence reads to a predetermined threshold to identify a probe-set-identifier value; (c) identifying the probe-set identifier based on the probe-set-identifier values of the corresponding regions; (d) generating, based on the probe-set identifier, a result indicating that the probe set includes one or more subject-specific capture probes that enrich the biological sample for a set of nucleic acid molecules of the plurality of nucleic acid molecules, wherein the set of nucleic acid molecules facilitate a determination of a classification of pathology for the subject; and (e) outputting the result.

In a further embodiment and in accordance with the above, the probe-set-identifier value is a binary value, and wherein identifying the probe-set identifier includes encoding the probe-set-identifier values.

In a further embodiment and in accordance with any of the above, the probe-set-identifier value is further identified by: (i) determining a first amount of sequence reads that align to a first region of the set of regions; (ii) determining a second amount of sequence reads that align to a second region of the set of regions; and (iii) comparing each of the first amount of sequence reads and the second amount of sequence reads to the predetermined threshold to identify the probe-set-identifier value.

In a further embodiment and in accordance with any of the above, identifying the probe-set identifier further includes: (i) identifying an erroneous probe-set-identifier value from the probe-set-identifier values of the set of regions; and (ii) modifying the erroneous probe-set-identifier value using a parity bit and/or an error correcting code.

In a further embodiment and in accordance with any of the above, the set of regions of the reference sequence correspond to a particular portion of a human genome.

In a further embodiment and in accordance with any of the above, the set of regions of the reference sequence correspond to genomic regions of a mitochondrial chromosome.

In a further embodiment and in accordance with any of the above, the set of regions of the reference sequence correspond to a particular portion of a non-human genome.

In a further embodiment and in accordance with any of the above, determining the amount of sequence reads that align to the region includes identifying a sequencing coverage for the region.

In a further embodiment and in accordance with any of the above, the method further comprises: (i) applying, to the biological sample, one or more additional capture probes to enrich the biological sample for nucleic acid molecules from another region; (ii) determining an amount of sequence reads that align to the other region; (iii) generating a normalization value based on the determined amount of sequence reads that align to the other region; and (iv) identifying the predetermined threshold based on the normalization value.

In a further embodiment and in accordance with any of the above, the set of self-identifying capture probes includes another self-identifying capture probe that enriches the biological sample for nucleic acid molecules from two or more regions of the set of regions, and wherein another probe-set-identifier value is identified based on an amount of sequence reads corresponding to each of the two or more regions.

In another aspect, the present disclosure provides a method comprising: (a) enriching a biological sample corresponding to a subject by applying, to the biological sample, a self-identifying capture probe of a probe set to enrich the biological sample for a first set of nucleic acid molecules of a plurality of nucleic acid molecules, wherein each nucleic acid molecule of the first set of nucleic acid molecules includes a first target sequence; and (b) sequencing the enriched biological sample to generate a set of sequence reads, wherein a subset of the set of sequence reads correspond to the first target sequence, wherein an amount of the subset of sequence reads represent an encoded probe-set-identifier value of a probe-set identifier of the probe set.

In a further embodiment and in accordance with the above, the probe-set identifier indicates whether the probe set is an expected probe set for determining a classification of pathology for the subject.

In another aspect, the present disclosure provides a method comprising: (a) enriching a biological sample corresponding to a subject by applying, to the biological sample, a self-identifying probe to enrich the biological sample for a set of nucleic acid molecules of a plurality of nucleic acid molecules, wherein the set of nucleic acid molecules facilitate a determination of a recent progression or a remission state of a disease of the subject, and wherein the set of nucleic acid molecules were identified by processing methylation data generated by processing a solid-tumor sample from the subject; (b) sequencing the enriched biological sample to generate a set of sequence reads; and (c) generating a result, using the set of sequence reads, that estimates a recent progression or remission state of the disease of the subject.

In another aspect, the present disclosure provides a system comprising: (a) one or more data processors; and (b) a non-transitory computer readable storage medium containing instructions which, when executed on the one or more data processors, cause the one or more data processors to perform part or all of one or more methods disclosed herein.

In another aspect, the present disclosure provides a computer-program product tangibly embodied in a non-transitory machine-readable storage medium, including instructions configured to cause one or more data processors to perform part or all of one or more methods disclosed herein.

In another aspect, the present disclosure provides a custom probe set comprising: a set of probes (e.g., including a HyperPETE, wherein the HyperPETE undergoes primer extension along a target of interest, hybrid capture probe, molecular inversion probe, or a normalization probe) that enrich a liquid biological sample for a first set of nucleic acid molecules, wherein the set of nucleic acid molecules facilitate a determination of a recent progression or a remission state of a disease of the subject, and wherein the set of nucleic acid molecules were identified by processing methylation data generated by processing a solid-tumor sample from the subject.

In a further embodiment and in accordance with the above, the set of probes comprises one or more of: (i) one or more HyperPETE, wherein each HyperPETE of the one or more HyperPETE undergoes primer extension along a target of interest, (ii) one or more hybrid capture probes, (iii) one or more molecular inversion probes, (iv) one or more self-identifying probes, (v) one or more normalization probes, or any combination thereof.

In another aspect, the present disclosure provides a custom probe set comprising: (a) a first set of capture probes that enrich the biological sample for a first set of nucleic acid molecules of the plurality of nucleic acid molecules, wherein the first set of nucleic acid molecules facilitate a determination of a classification of pathology for the subject; and (b) a second set of capture probes that enrich the biological sample for a second set of nucleic acid molecules of the plurality of nucleic acid molecules, wherein a measured amount of the second set of nucleic acid molecules encodes a probe-set-identifier value of a probe-set identifier of the custom probe set.

In some embodiments, a computer-implemented method is provided. Sequencing data is accessed that had been generated by sequencing a processed sample obtained from a subject, the sequencing data including or having been based on a set of sequence reads. Using the sequencing data, one or more loci are identified that correspond to single nucleotide polymorphisms (SNPs) at which at least a threshold number or percentage of the set of sequence reads included a base identifier that departed from a reference base identifier corresponding to a same position in a reference sequence. For each locus of the one or more loci and for each of one or more positions within a sequence portion that includes the locus, a methylation percentage is determined using reads that include the corresponding SNP. For each locus of the one or more loci and for each of the one or more positions corresponding to the sequence portion that includes the locus, a comparative methylation percentage is identified. A result is generated based on each determined methylation percentage and each comparative methylation percentage, where the result represents a prediction as to whether the sample is associated with a particular medical condition, whether the sample is associated with a medical condition of a particular stage, whether the subject has a particular type of medical condition, or whether the sample was collected from a specific individual. The result is output.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by some embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Fig.,” “FIG.,” “Figure,” “Figures,” “Figs.,” and “FIGs.” herein) of which:

FIG. 1 shows an example of a process for classifying a read according to some embodiments.

FIG. 2 shows an example of a process for classifying a read according to some embodiments.

FIG. 3 shows a schematic diagram illustrating a process for targeted enrichment of a biological sample, according to some embodiments.

FIG. 4 shows a flowchart illustrating an example of a method of assigning a probe-set identifier of a corresponding probe set, according to some embodiments.

FIG. 5 shows an example of a schematic diagram for determining a probe-set identifier of a probe set, according to some embodiments.

FIG. 6 shows a flowchart illustrating an example of a method of determining a probe-set identifier of a corresponding probe set, according to some embodiments.

FIG. 7 shows an example of a computer system for implementing some embodiments.

FIG. 8 shows a plot of an expected probability of detection versus tumor fraction-both when methylation is considered in addition to bases (so as to indicate any single nucleotide polymorphisms) and when only bases are considered.

FIG. 9 shows a circumstance where a normal sequence includes normal cells with an unmethylated CpG site and a thymine and tumor cells with a methylated CpG site and a guanine.

FIG. 10 shows a circumstance where a normal sequence includes normal cells with multiple unmethylated CpG sites and tumor cells with multiple methylated CpG sites.

### DETAILED DESCRIPTION OF THE INVENTION

### 1. Overview

Sequencing data that is accessed may have been generated by processing a sample from a subject. The sample may include a liquid sample (e.g., a blood sample) and/or a sample including cell-free DNA. The sample includes a plurality of nucleic acid molecules. The nucleic acid molecules can be deoxyribonucleic acid (DNA), ribonucleic acid (RNA) or any hybrid or fragment thereof. The nucleic acid in the sample may be a cell-free nucleic acid. In some instances, the biological sample includes a mixture of cell-free nucleic acid molecules from the subject and potentially nucleic acid molecules from a pathogen, e.g., a virus. For example, the biological sample can include circulating tumor DNA (ctDNA) or circulating tumor RNA (ctRNA).

Additionally, or alternatively, the biological sample can include any tissue or material derived from a subject. For example, the biological sample can include a core needle biopsy sample or a fine needle aspirate biopsy sample. The biological sample may be a liquid sample or a solid sample (e.g., a cell or tissue sample). In some cases, the biological sample may be from a sentinel lymph node or an auxiliary lymph node dissection. The nucleic acid molecules can be obtained from circulating tumor cells in the biological sample.

The sequencing data can include a set of sequence reads that had been generated by sequencing the sample. Each of the set of sequence reads can be aligned to a reference sequence. In some embodiments, the reference sequence is a generic human reference sequence, such as, for example, Hg18 or Hg19. In some preferred embodiments, the reference sequence is a normal human reference sequence of the subject. In certain embodiments, the use of a normal human reference sequence of the subject provides superior technical advantages (such as, for example, an increase in signal detection over a noise floor) when compared to a method that utilizes a generic human reference sequence. However, in other certain embodiments, the use of a generic human reference may be technically advantageous when compared to a method that utilizes a subject-specific normal reference. Further, in other certain embodiments, the use of a population-level human reference sequence or a human reference sequence generated from a plurality of individuals may demonstrate superior technical properties compared to the use of a generic human reference sequence or a normal human reference sequence of the subject (such as, for example, in circumstances where a sufficient number of genetic parameters (e.g., polymorphisms, methylation state, etc.) cannot be determined using a reference sequence from a singular subject). Still further, in some embodiments, it may be advantageous to use a combination of reference sequences, wherein at least a first subset of the sequencing data is aligned using a first reference sequence (such as, for example, population-level sequencing data, sequencing data derived from one or more databases, etc.) and at least a second subset of the sequencing data is aligned using a second reference sequence (such as, for example, sequencing data generated from a “normal” sample of a subject). In some instances, the alignment includes determining whether multiple bases (or sets of bases) are duplicative and removing the duplicate base(s). One or more pieces of software and/or toolkits, such as (for example) the Picard toolkit (RRID:SCR_006525) and/or Genome Analysis Toolkit (e.g., GATK, RRID:SCR_001876) may be used for the alignment. Aligned sequence data may be returned in BAM format according to the SAM (RRID:SCR_01095) specification. In some instances, the bases of a read are identical to bases in a portion of the reference sequence to which the read is aligned. In other instances, there are one or more differences between bases of the read and bases of the portion of the reference sequence to which the read is aligned. These differences may be characterized or identified as one or more variants. A difference of a single base identifier is characterized as a single nucleotide polymorphism (SNP).

In instances where a sample includes both normal cells and tumor cells, each read in an incomplete subset of the reads aligned to a portion of the reference sequence may include a variant. For example, if 10 reads include an identifier of a base that is aligned to a particular position, 8 “normal” reads may include a base identifier that is the same as one in a reference sequence, while 2 “tumor” reads may include a different base identifier. One problem is that sequencing errors may also result in inaccurate base identifications. Thus, if a base identifier is different than a corresponding base identifier in a reference sequence, it may be due to an actual variant (e.g., a SNP) or due to a sequencing error. If a substantial portion of a sample is from a tumor, it becomes easier to detect variants of the tumor. However, detecting whether a subject has a disease when a very small portion of the DNA in a sample is from a tumor is more challenging. Similarly, detecting precise proportions of a sample that are cancerous can also be difficult due to noise challenges.

In some embodiments, methylation signals are used to facilitate classifying each of various portions of sequencing data. For example, one or more methylation signals from each read may be classified as corresponding to a sequence from (e.g., that had been released from) a normal cell versus a sequence from a diseased cell (e.g., a cancer cell). As another example, one or more methylation signals from each read with a distinction from an aligned portion of a reference sequence can be classified as being from a diseased cell or having an inaccurate base identifier generated based on a sequencing error. A methylation signal may correspond to a base that is a SNP variant or a base that is within a predefined range of bases from a SNP. For example, if it is determined that a cytosine that precedes a SNP by 3 bases is methylated in reads with the SNP, whereas the cytosine that precedes a corresponding non-SNP base by 3 bases is not methylated, consistent co-occurrence of the methylation and the SNP in individual reads can multiplicatively decrease the probability that the methylation or SNP occurred due to a sequencing error, whereas the probability decrease in instances where each of two reference-sequence departures were observed in different reads may be additive in nature.

In some instances, methylation percentages can be determined and evaluated for any cytosine in a CpG region and/or for any cytosine in a CpG region where a given condition is satisfied (e.g., having at least a threshold number of reads aligned for the region). This approach may be used (for example) to perform a personalized assay to monitor an individual subject's disease state. In some instances, methylation data is selectively evaluated for CpG regions for which reference data indicates that a “normal” methylation percentage is above a given upper threshold (e.g., 80%, 85%, 90% or 95%) or below a given lower threshold (e.g., 20%, 15%, 10% or 5%). To determine these normal methylation percentages, a data source may be used, such as UCSC Genome Browser (Karolchik D, Hinrichs AS, Furey TS, Roskin KM, Sugnet CW, Haussler D, Kent WJ. The UCSC Table Browser data retrieval tool. Nucleic Acids Res. 2004 Jan. 1; 32 (Database issue):D493-6), MethBase data tracks (Song Q, Decato B, Hong E, Zhou M, Fang F, Qu J, Garvin T, Kessler M, Zhou J, Smith AD (2013) A reference methylome database and analysis pipeline to facilitate integrative and comparative epigenomics. PLOS ONE 8(12): e81148), or TCGA (The Cancer Genome Atlas database, which is available at https://www.cancer.gov/about-nci/organization/ccg/research/structural-genomics/tcga), each of which is incorporated by reference for all purposes. This approach may be particularly advantageous when predicting whether a subject has cancer generally or a particular type of cancer.

Thus, some embodiments include detecting each SNP that occurs within at least a threshold number or percentage of reads aligned to a corresponding position and evaluating—for each read that contains the SNP—a methylation state at each of one or more positions (e.g., predefined positions) that are within a predefined distance upstream or downstream from the SNP. For each of these positions, a methylation percentage can be calculated as the number of reads that include both the SNP and a methylated base at the position divided by the number of reads that include the SNP. A comparative methylation percentage may indicate a likelihood of a methylated base being present at the position in normal reads (that do not include the SNP). The comparative methylation percentage may be determined using a look-up table (e.g., generated using sequence data from one or more other subjects) or by using reads in the subject's sequencing data that do not include the SNP (but are aligned to a region that includes a position corresponding to the SNP). Alternatively, or in addition to, the comparative methylation percentage may be determined using a look-up table generated using population-level sequencing data (or, in some instances, population-level methylation data) and/or by using sequence reads in the subject's sequencing data generated from a “normal” sample (or, in instances where a sample comprises both normal nucleic acids and tumor-derived nucleic acids, using sequence reads from the “normal” portion). Alternatively, or in addition to, the comparative methylation percentage may be determined using a combination of population-level sequencing data (e.g., population-level methylation data) and sequence reads in the subject's sequencing data generated from a “normal” sample (or, in instances where a sample comprises both normal nucleic acids and tumor-derived nucleic acids, using sequence reads from the “normal” portion). Phrased differently, in some embodiments, a combination of population-level sequencing data and subject-specific normal sequencing data may be used to determine the comparative methylation percentage (i.e., a first subset of the comparative methylation percentage is determined using at least part of the population-level sequencing data, and a second subset is determined using at least part of the subject-specific normal sequencing data). In some instances, the subject-specific normal sequencing data was generated prior to or separately from the methods of the disclosure. In other instances, the subject-specific normal sequencing data can be generated simultaneously and/or sequentially with subject-specific tumor sequencing data. A difference between the methylation percentage and comparative methylation percentage can serve as a biomarker for the tumor and/or can support a conclusion that the reads with the SNP truly include a variant and that the base difference of the SNP is not just due to a sequencing error.

In some instances, one or more population data sets (e.g., generated based on sequences of samples collected from multiple other subjects) can be used to identify one or more pan-cancer methylation biomarkers (corresponding to many different cancers of different tumor origins) or one or more cancer-specific methylation biomarkers (e.g., corresponding to a specific tumor-origin anatomical location, or corresponding to a specific cancer stage), etc.

In some instances, one or more population data sets can be used in conjunction with one or more subject-specific data sets (i.e., nucleic acid sequencing data generated from sequencing one or more samples from a subject) to identify one or more pan-cancer methylation biomarkers, one or more cancer-specific methylation biomarkers, one or more subject-specific methylation biomarkers, etc.

Some embodiments include using a solid-tumor sample that was collected from a subject to generate a tumor-sequence signature that can then be used to detect reads corresponding to the tumor in a cell-free sample. For example, the sample can include a core needle biopsy sample or a fine needle aspirate biopsy sample. Sequence reads generated by processing a solid tumor can be aligned to a reference sequence and used to identify both the sequence of the tumor and methylation percentages at different positions. The sequence of the tumor and the methylation percentages can be compared to those from a comparative sequence (e.g., a sequence generated by processing a non-tumor sample of the subject or a reference sequence generated by processing one or more samples from one or more other subjects). Each distinction between a base in the solid-tumor sequence and a corresponding base in a comparative sequence can be defined as a biomarker for the tumor and/or a part of a signature for the tumor. Each distinction between a methylation percentage for a position (e.g., a locus) in the solid-tumor reads and a comparative methylation percentage for the position can be defined as a biomarker for the tumor and/or a part of a signature for the tumor.

Thus, a difference between a base in a read and a corresponding base in a reference sequence (e.g., a SNP) can be a biomarker for a cancer and a given methylation state (e.g., methylated or not) can be a biomarker for a cancer. When multiple biomarkers are present in a given read, the probability of the read corresponding to DNA from a tumor may be multiplicatively or exponentially higher than if the read included only one biomarker. Thus, a tumor-sequence signature that includes methylation biomarkers (e.g., potentially in addition to variant biomarkers) can improve the precision, recall, specificity and/or sensitivity of accurately classifying a read as a tumor or normal read. More accurate detection of tumor reads can help more accurately predict whether and/or a degree to which a subject's disease is progressing (or alternatively remitting). This information may inform a treatment selection or characteristic of a treatment regimen (e.g., frequency of treatment administrations).

In some instances, a probe set can be provided to enrich the sample for a first set of nucleic acid molecules. In some embodiments, the probe set comprises a self-identifying capture probe set, as further described herein. Each nucleic acid molecule of the first set of nucleic acid molecules includes a first target sequence. The first target sequence can correspond to a sequence with a methylation biomarker (e.g., potentially in addition to a variant). The probe may include a hybridization capture probe, one or more HyperPETE (wherein each HyperPETE of the one or more HyperPETE undergoes primer extension along a target of interest), a hybrid capture probe, a self-identifying capture probe, or a molecular inversion probe. In some further instances, the probe set can further comprise capture probes to be used for normalization of sequencing data, genomic region(s) of interest, etc.

A first amount of the first set of nucleic acid molecules can be determined. In some instances, the first amount of the first set of nucleic acid molecules can be determined by: sequencing the plurality of nucleic acid molecules of the enriched biological sample to obtain a plurality of sequence reads; aligning each of the plurality of sequence reads to a corresponding portion of a human reference genome (e.g., a generic human reference genome, a subject-specific reference genome generated from a “normal” sample, a generic human reference genome generated from a plurality of individuals, a generic human reference genome generated from population-level data, etc.); identifying, from the aligned sequence reads, a set of sequence reads that correspond to the first target sequence; determining an amount of the set of sequence reads; and identifying, based on the amount of the set of sequence reads, a sequencing coverage for the probe set.

A probe-set identifier of the probe set can then be identified based on the first amount of the first set of nucleic acid molecules. In some instances, the probe-set identifier is identified based on determining whether the sequencing coverage exceeds a predetermined threshold. If the sequencing coverage exceeds the predetermined threshold, a first value of the probe-set identifier can be determined, in which the first value is predictive of a presence of the first target sequence in the biological sample. In contrast, if the sequencing coverage does not exceed the predetermined threshold, a second value of the probe-set identifier can be determined, in which the second value is predictive of an absence of the first target sequence in the biological sample.

The probe-set identifier can be used to generate a result indicating that the probe set is specifically designed to analyze the sample. In particular, the result indicates that the probe set includes one or more subject-specific capture probes that enrich the biological sample for a second set of nucleic acid molecules of the plurality of nucleic acid molecules. The second set of nucleic acid molecules facilitate a determination of a classification of pathology for the subject. In some instances, the pathology corresponds to cancer such as hepatocellular carcinoma. Custom assays, such as the probe set that includes the one or more subject-specific capture probes, can thus be correctly selected and used for identifying and tracking genetic mutations in the subject. Details of developing the custom assays are provided in U.S. Pat. No. 10,450,611, which is incorporated herein by reference in its entirety for all purposes.

In some instances, varying laboratory conditions, including variations in time allowed for hybrid capture, temperature at which the hybridization is conducted, or amplification before or after capture are considered, such that the probe-set identifier can be consistently and correctly determined. This includes applying a normalizing capture probe of the probe set to enrich the biological sample for a third set of nucleic acid molecules of the plurality of nucleic acid molecules, in which each nucleic acid molecule of the third set of nucleic acid molecules includes a second target sequence.

A second amount of the third set of nucleic acid molecules can be determined, and the second amount can be used to determine or otherwise adjust the threshold that is being used to compare against the sequencing coverage. Additionally, or alternatively, a statistical value can also be determined based on the second amount and identifying the probe-set identifier based on the statistical value.

Other variations on this approach can also be clear to those skilled in the art. For example, instead of using hybrid capture probes, primers/amplicons can be used instead. Similar to capture probes, the amplicon-based assay specifically can create a sequencing coverage profile which can be interpreted into a custom-assay identifier, without needing to compare those results with an assay design database. In the case of an amplicon panel, the information content of each coverage peak of the sequencing coverage plot can generate a two-dimensional code space, derived from the two primers of the amplicon. This is similar to having a pair of hybrid capture probes in a target genomic region. Such implementation can create a two-dimensional code space for identifying the assay identifier. Such code space can include multiple bits of information which contribute to identifying the assay identifier from the sequencing coverage plot.

In some instances, a sample (e.g., that includes cell-free DNA) is enriched such that subsequent sequencing can be targeted towards select regions within a genome. The select regions can include a methylation biomarker (e.g., identified based on sequences from a solid-tumor sample). The enriched sample can then be sequenced, and each sequence read can be classified as a tumor read or normal read using a technique disclosed herein.

The following examples are provided to introduce certain embodiments. In the following description, for the purposes of explanation, specific details are set forth in order to provide a thorough understanding of examples of the disclosure. However, it will be apparent that various examples may be practiced without these specific details. For example, devices, systems, structures, assemblies, methods, and other components may be shown as components in block diagram form in order not to obscure the examples in unnecessary detail. In other instances, well-known devices, processes, systems, structures, and techniques may be shown without necessary detail in order to avoid obscuring the examples. The figures and description are not intended to be restrictive. The terms and expressions that have been employed in this disclosure are used as terms of description and not of limitation, and there is no intention in the use of such terms and expressions of excluding any equivalents of the features shown and described or portions thereof. The word “example” is used herein to mean “serving as an example, instance, or illustration.” Any embodiment or design described herein as an “example” is not necessarily to be construed as preferred or advantageous over other embodiments or designs.

### II. Definitions

Unless defined otherwise, technical, and scientific terms used herein have the same meaning as commonly understood by one of ordinary skill in the art to which the present disclosure belongs. For purposes of the present disclosure, the following terms are defined below. The definitions provided are intended to apply to a given term, as well as other derivative linguistic re-phrasings and grammatical equivalents of the term.

As used herein, the term “subject” generally refers to any organism that is used in the methods of the disclosure. In some examples, a subject is a human, mammal, vertebrate, invertebrate, eukaryote, archaea, fungus, or prokaryote. In some instances, a subject can be a human. A subject can be living or dead. A subject can be a patient. For example, a subject may be suffering from a disease (or suspected of suffering from a disease) and/or in the care of a medical practitioner. A subject can be an individual that is undergoing treatment and/or diagnosis for a health or medical condition. A subject and/or family member can be related to another subject used in the methods of the disclosure (e.g., a sister, a brother, a mother, a father, a nephew, a niece, an aunt, an uncle, a grandparent, a great-grandparent, or a cousin).

As used herein, the term “example” is used herein to mean “serving as an example, instance, or illustration.” Any embodiment or design described herein as an “example” is not necessarily to be construed as preferred or advantageous over other embodiments or designs.

As used herein, the term “methylation percentage” includes an estimate of a percentage of bases that are methylated, an estimate of a fraction of bases that are methylated, an estimate of a probability that a base is methylated, or any other statistic that can be used to estimate a methylation prevalence for a given position.

As used herein, the term “amplification” refers to any process of producing at least one copy of a nucleic acid molecule. The terms “amplicons” and “amplified nucleic acid molecule” refer to a copy of a nucleic acid molecule and can be used interchangeably. The amplification reactions can comprise PCR-based methods, non-PCR based methods, or a combination thereof. Examples of non-PCR based methods include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. PCR-based methods may include, but are not limited to, PCR, HD-PCR, Next Gen PCR, digital RTA, or any combination thereof. Additional PCR methods include, but are not limited to, linear amplification, allele-specific PCR, Alu PCR, assembly PCR, asymmetric PCR, droplet PCR, emulsion PCR, helicase dependent amplification HDA, hot start PCR, inverse PCR, linear-after-the-exponential (LATE)-PCR, long PCR, multiplex PCR, nested PCR, hemi-nested PCR, quantitative PCR, RT-PCR, real time PCR, single cell PCR, and touchdown PCR.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

Conditional language used herein, such as, among others, “can,” “could,” “might,” “may,” “e.g.,” and the like, unless specifically stated otherwise, or otherwise understood within the context as used, is generally intended to convey that certain examples include, while other examples do not include, certain features, elements, and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Similarly, the use of “based at least in part on” is meant to be open and inclusive, in that a process, step, calculation, or other action “based at least in part on” one or more recited conditions or values may, in practice, be based on additional conditions or values beyond those recited. Headings, lists, and numbering included herein are for ease of explanation only and are not meant to be limiting.

As used herein, the singular forms “a,” “an,” and “the” include plural referents unless the context clearly dictates otherwise. Thus, for example, reference to “an antigen” includes mixtures of antigens; reference to “a pharmaceutically acceptable carrier” includes mixtures of two or more such carriers, and the like. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A (alone),” and “B (alone).”

As used herein, the term “about” a value (or parameter) refers to ±10% of a stated value. When referring to a range of values (or parameters), the term “about” refers to +10% of the upper limit and −10% of the lower limit of a stated range of values. When a range of values is provided, it is to be understood that each intervening value between the upper and lower limit of that range, and any other stated or intervening value in that stated range, is encompassed within the scope of the present disclosure. Where the stated range includes upper and/or lower limits, ranges excluding either of those included limits are also included in the present disclosure.

The various features and processes described above may be used independently of one another or may be combined in various ways. All possible combinations and sub-combinations are intended to fall within the scope of the present disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed examples. Similarly, the example systems and components described herein may be configured differently than described. For example, elements may be added to, removed from, or rearranged compared to the disclosed examples.

### III. Exemplary Process for Facilitating Read Classification Using Population-Level and Sample Methylation Data

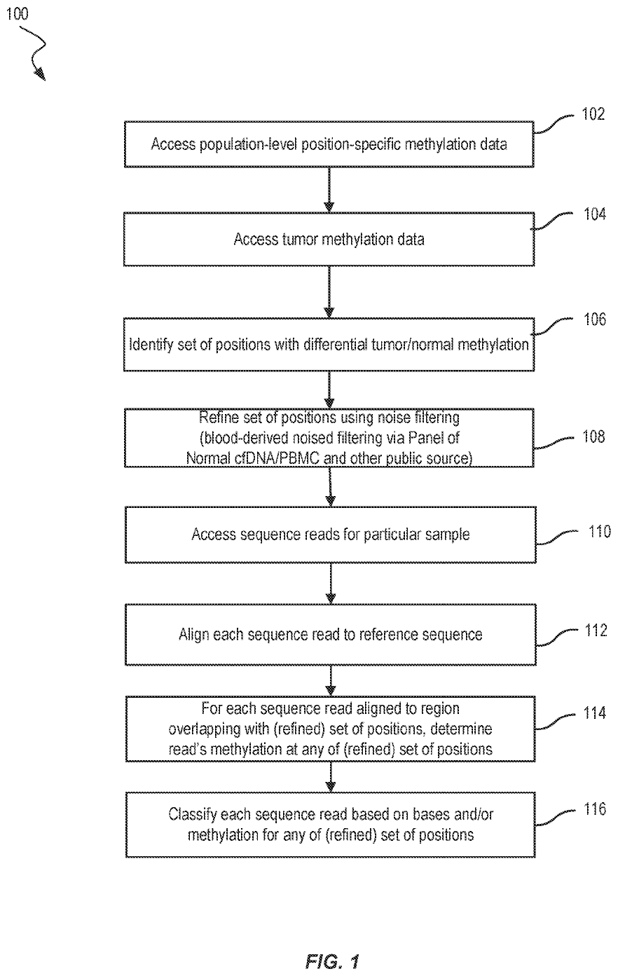

FIG. 1 illustrates a process 100 for classifying a read according to some embodiments of the present invention. Process 100 begins at block 102, where population-level methylation data is accessed. For each position or locus within part or all of a reference genome, the population-level methylation data may indicate what percentage or fraction of bases (from various reads) aligned to the specific position are methylated. The population-level methylation data may be generated using sequencing data generated by processing samples from multiple individuals, e.g., where each of the multiple individuals had been identified or determined as being healthy, not having any disease, not having cancer, or not having a particular type of cancer. Thus, the population-level methylation data can be characterized as identifying “normal” methylation percentages. Block 102 may include generating the population-level methylation data or retrieving the population-level methylation data from a source.

In some instances, a methylation percentage is calculated for each of multiple positions for each of the multiple individuals, and those methylation percentages are averaged to generate the methylation percentage in the population position-specific methylation data (e.g., so as to adjust to different coverages across individuals). As used herein, a “methylation percentage” includes an estimate of a percentage of bases that are methylated, an estimate of a fraction of bases that are methylated, an estimate of a probability that a base is methylated, or any other statistic that can be used to estimate a methylation prevalence for a given position.

The population-level position-specific methylation data may identify the methylation fraction for only some loci or only some positions within a genome of part or a genome (e.g., one or more chromosomes or one or more genes). The some loci may include positions where a cytosine nucleotide from a CpG site is aligned. In some instances, the population-level position-specific methylation data may not contain information for a given region of interest. In such a situation, it may be advantageous to access subject-specific methylation data to determine the “normal” methylation status of the given region of interest.

At block 104, tumor methylation data is accessed. The tumor methylation data may be generated using one or more diseased samples. Because a diseased sample may include both normal and tumor DNA, the tumor methylation data may include methylation data identified by analyzing reads or fragments that include a variant. The tumor methylation data may identify—for each of a set of loci—a probability that a base (e.g., a cytosine) aligned to the locus is methylated

The tumor methylation data may be specific to a particular subject, a particular type of cancer, a particular stage of cancer, cancer generally, etc. For example, the tumor methylation data may have been generated by, for each of a set of subjects diagnosed as having a particular type of cancer, processing a diseased sample to generate a set of reads, aligning the reads to a reference sequence (which may, but need not, be a reference sequence corresponding to the population-level position-specific methylation data), and estimating—for each of a set of loci—a methylation percentage based on how many bases aligned to the locus were methylated. When the tumor methylation data is not subject-specific, methylation percentages may instead be generated by calculating a preliminary methylation percentage for each of multiple subjects (e.g., who have a particular disease) and then calculating an average or median of the percentages across subjects.

In an instance where the tumor methylation data is specific to a particular subject, it may be unknown-as of a time at which the sample is assessed-whether the sample is a diseased sample (e.g., whether the sample includes tumor cells). Thus, it will be appreciated that while some disclosures herein may refer to a “diseased sample,” “tumor methylation data,” etc., it may not yet be known whether the particular subject and/or sample has a disease or tumor cells. In some instances, a result of process 100 may actually include a prediction that the particular subject does not have cancer, does not have a one or more diseases, etc. In some instances where the diseased sample includes both normal and tumor DNA, it may be advantageous to access a combination of population-level methylation data and subject-specific methylation data to facilitate discriminating between sequence reads from the normal DNA and tumor DNA.

A technique that can be used to investigate methylation can include using (for example) methyl-converted sequencing, corresponding to (for example) sequencing performed after bisulfite conversion, enzymatic, or other conversion techniques. The sequencing may include direct sequencing, which may include direct sequencing of some or all bases known or predicted to be methylated in at least a portion of reference sequences. Direct sequencing may use (for example) PacBio, NovaSeq, PacBio RS, RSII, Sequel, Sequel II, Element Biosciences Aviti, Genapsys, Oxford Nanopore or other sequencing platforms configured to output a readout of which bases are methylated The sequencing may use array or bead hybridization, a bead array, PCR (e.g., to amplify methyl-converted DNA, where PCR may include, for example, quantitative PCR, or digital droplet PCR), methylation-specific PCR, pyrosequencing, etc. The technique may include target sequencing, which may occur pre-conversion or post-conversion (e.g., when using methyl-converted DNA). For pre-conversion target sequence, capture probes may be based on specific genomic loci suspected to be methylated in non-diseased instances (e.g., based on a reference genomic sequence). The capture probes may comprise self-identifying capture probes. A conversion protocol may then be implemented to (for example) selectively convert the captured sequences.

It will be appreciated that various tools and/or techniques may be used to make and/or use embodiments of the invention disclosed herein. Exemplary techniques and/or tools may be configured (for example) to remove adaptor sequences, to remove low quality 3′ ends, for read alignment, to quantify methylation context, to quantify level extractions, to group UMIs, to perform PCR (e.g., methylation-specific PCR), to apply probes (e.g., methylation-specific probes), to apply primers (e.g., methylation-specific primers), to mark PCR duplications, to remove PCR duplications, for library and/or enrichment quality-control metrics, to sort bam files, to format methylation call outputs, to sort and convert aligned SAM files to BAM files, to index BAM files, to enumerate variant and/or methylation supporting reads, to extract methylation context and/or levels, and/or to convert unmethylated cytosine residue to uracil (e.g., using a chemical agent such as a bisulfite salt or using an enzymatic conversion). Such techniques and/or tools used to support embodiments of the invention may include a technique and/or tool as disclosed in: U.S. Pat. No. 10,590,468 B2; Lee, I., Razaghi, R., Gilpatrick, T. et al. “Simultaneous profiling of chromatin accessibility and methylation on human cell lines with nanopore sequencing.” Nat Methods 17, 1191-1199 (2020). https://doi.org/10.1038/s41592-020-01000-7; and/or Romualdas Vaisvila, V. K. Chaithanya Ponnaluri, et al. “EM-seq: Detection of DNA Methylation at Single Base Resolution from Picograms of DNA” bioRxiv 2019.12.20.884692; doi: https://doi.org/10.1101/2019.12.20.884692, each of which is incorporated by reference in its entirety for all purposes.

At block 106, a set of positions (or a set of loci) are identified where a methylation percentage from the normal methylation data sufficiently differs from a methylation percentage from the tumor methylation data. Block 106 may include performing a statistical test to predict a likelihood that any observed difference between a methylation percentage from the normal methylation data and a methylation percentage from the tumor methylation data occurred due to chance. Block 106 may include calculating a p-value based on just two numbers: a position-specific methylation percentage from the normal methylation data and a position-specific methylation percentage from the tumor methylation data. Alternatively, block 106 may include generating a distribution or statistical value (e.g., variance, standard deviation and/or mean) based on multiple methylation percentages from the normal methylation data and using the distribution or statistical value in combination with the position-specific methylation percentage from the normal methylation data and the position-specific methylation percentage from the tumor methylation data to generate a p value. The set of positions may be identified as those positions where a p value is below a predefined threshold (e.g., 0.1, 0.05, 0.01, or 0.001). In some instances, rather than or instead of identifying a set of positions with differential methylation at block 106, multiple positions (e.g., for which a cytosine nucleotide from a CpG site was aligned in a reference sequence) are ordered based on a degree of difference, a p-value, etc.

In some instances, block 106 includes implementing a processing configuration that ensures that each of the set of positions that are identified are within a predefined distance (e.g., within 50 bases, within 20 bases, within 10 bases, within 5 bases, etc.) from a SNP in tumor sequencing data that corresponds to the tumor methylation data. For example, a statistical analysis may be configured to selectively perform a statistical test for such regions within a sequence. In some instances, the normal methylation data accessed at block 102 or the tumor methylation data accessed at block 104 only includes methylation data for such regions.

At block 108, the set of positions is refined using noise filtering. More specifically, sequencing and securing methylation data are each error-prone processes. Thus, it is possible that a result that indicates that a diseased sample has a particular variant. or a particular methylation distinction (relative to normal), is erroneous. The chances of such an error are lower the more reads for which the variant was observed or for which the methylation distinction was observed. The chances of such an error are also lower when individual reads include more than one difference relative to normal data (e.g., a variant and also one or more methylation distinctions).

The noise filtering can be configured to estimate whether a detected variant or a detected methylation distinction is likely to be due to a sequencing error. The noise filtering may be based on data that indicates or that can be used to predict a likelihood that one or more distinctions (e.g., including one or more variants and/or one or more methylation distinctions) that were detected within a given region (e.g., within a genome or within a particular gene) occurred by chance. For example, suppose that 20 sequence reads were aligned so as to completely overlap with the given region. Suppose that 3 of the sequence reads included a same base departure (at a same position) relative to a reference sequence and that 2 of those sequence reads included a methylated cytosine within the region (where only 1 of the other 17 sequence reads included a corresponding methylated cytosine and the remaining 16 included an unmethylated cytosine). In this example, block 108 can include looking up a likelihood of the base departure being present in a sequence read from a normal sample and looking up a likelihood of the cytosine being methylated (e.g., presumably due to a sequencing error). Such information may be or may have been generated by using (for example) a Panel of Normal cfDNA or peripheral blood mononuclear cells (from one or more normal samples). This analysis may be performed by evaluating multiple distinctions co-occurring. For example, rather than first evaluating a likelihood that a given base departure occurred due to a sequencing error and then evaluating a likelihood that a given discrepancy in a methylation percentage occurred due to a sequencing error, the evaluation may include evaluating the likelihood that a sequencing error resulted in both the base departure and the methylation-percentage discrepancy (e.g., in the same reads).

At block 108, the set of positions can be refined to exclude positions where it has been determined that a methylation-percentage discrepancy is likely due to a sequencing error (and not due to a disease). In some instances, instead of or in addition to excluding one or more positions, block 108 includes assigning a weight to each of the set of positions that is based on a likelihood that a discrepancy at that position would have occurred due to a sequencing error. In some instances, instead of or in addition to excluding one or more positions, block 108 includes assigning a weight to a region that is based on a likelihood that a combination of discrepancies at each of two or more positions (of the set of positions) within the region include a discrepancy at that position.

At block 110, a set of sequence reads that were generated by processing a sample is accessed. The particular sample may include a diseased sample or a sample from an individual for which it is not known whether the individual has a particular disease (e.g., cancer) or for which it is not known whether a particular disease (e.g., cancer) is remitting, progressive, or in between. The particular sample may include a blood sample and/or a sample with cell-free DNA.

At block 112, each of the set of sequence reads is aligned to a reference sequence. At block 114, for each sequence read that is aligned to a region that overlaps with at least one of the set of positions (or refined set of positions), a methylation state is determined for each of any of the set of positions (or refined set of positions) within the read.

At block 116, each sequence read is classified using the bases in the read and/or the methylation state of any of the set of positions (or refined set of positions) corresponding to the sequence read. For example, a classification using the bases in the read may be based on whether a base in the read differs from a corresponding reference read (and/or is a SNP). As another example, a classification using the methylation state may be based on a corresponding normal methylation percentage, a tumor methylation percentage and/or the methylation state. The classification may depend on a likelihood that a given base discrepancy or methylation discrepancy was due to a sequencing error. The classification may depend on a weight assigned to one or more of the set of positions. The classification may be performed using a machine-learning model, such as a clustering model. In some instances, in addition to classifying each read, a confidence metric is also defined for each classification.

While not shown, the classifications of individual reads can then be used to predict whether a subject (corresponding to the particular sample) has a given disease, whether a disease of the subject is in remission, whether a disease of the subject is progressing, whether a recent treatment administered to the subject is estimated as being effective, etc. Such predictions may depend on classifications of multiple reads and potentially also confidence metrics corresponding to the classifications. As indicated herein, such predictions may influence a diagnose and/or treatment decision.

### IV. Exemplary Process for Facilitating Read Classification Using Disease and Cell-free Methylation Data

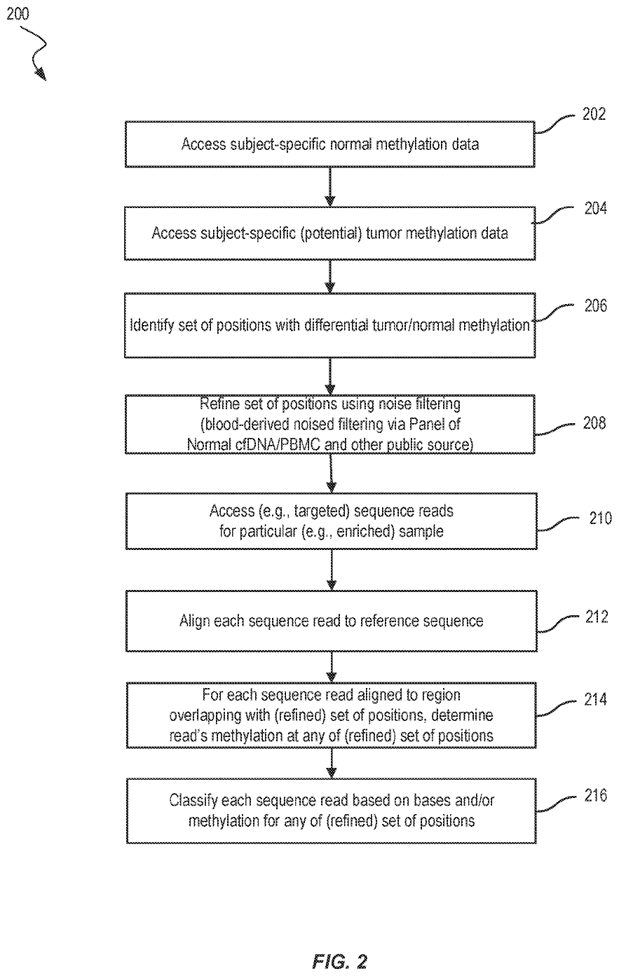

FIG. 2 illustrates a process 200 for classifying a read according to some embodiments of the present invention. Many of the actions in process 200 are similar to or the same as corresponding actions in process 1100. However, in process 200, the normal methylation data accessed at block 202 is subject-specific. While exemplary processes are set forth for embodiments that separately access subject-specific methylation data or population-level methylation data, it is expressly contemplated that, in certain embodiments, it may be technologically advantageous to access a combination of the subject-specific methylation data and population-level methylation data. Phrased differently, in some embodiments, the normal methylation data accessed at block 202 can comprise subject-specific methylation data and population-level methylation data. Thus, the normal methylation data can be generated using a sample that is known or believed not to be diseased (e.g., due to being from a part of the body that is different from a part of the body that is known or suspected to be diseased and/or due to the subject not having been previously diagnosed with cancer). The different part of the body may be an adjacent part of the body. For example, if a subject is being investigated for potentially having a cancer of the liver (e.g., cholangiocarcinoma), a sample collected to generate the (potential) tumor methylation data at block 204 may include a biopsy from the liver, whereas a sample collected to generate the normal methylation data at block 202 may include a cancer from the pancreas. This approach can capitalize on methylation patterns being tissue-type-specific, and further—even within a same type of tissue—methylation patterns differing between diseased and normal sample. For example, The Cancer Genome Atlas database (which is available at https://www.cancer.gov/about-nci/organization/ccg/research/structural-genomics/tcga) includes matched adjacent normal methylation data from a variety of tissue types, based on results generated by using the Illumina 450 array and/or using a technique as disclosed in Moss, J., Magenheim, J., Neiman, D. et al. Comprehensive human cell-type methylation atlas reveals origins of circulating cell-free DNA in health and disease. Nat Commun 9, 5068 (2018). https://doi.org/10.1038/s41467-018-07466-6 (available at https://www.nature.com/articles/s41467-018-07466-6 #Sec13, which is hereby incorporated by reference in its entirety for all purposes).

Methylation patterns of a normal sample may thus be used to identify a tissue of origin for a sample. To illustrate, if a subject is being investigated for potentially having a cancer of the liver (e.g., cholangiocarcinoma), a sample collected to generate the (potential) tumor methylation data at block 204 may include a biopsy from the liver, whereas a sample collected to generate the normal methylation data at block 202 may include a normal tissue biopsy from the liver. (This example would thus be using a tumor/normal tissue pair from a same subject.)

Thus, in process 200, the predictions as to what types, positions, and/or extents of discrepancies (e.g., a single-base discrepancy, binary methylation discrepancy, or methylation-percentage discrepancy) may be performed based on reference data that is specific to the same subject from whom a sample from which the (potential) tumor methylation data was generated.

In some instances, an individual may innately have (or may have acquired having) a variant and/or methylation-percentage discrepancy. While a population-level evaluation of normal methylation data (e.g., corresponding to process 100) may provide an informative baseline of a likelihood that an observed discrepancy is representative of a disease in a sample, using a reference that is subject-specific may potentially be even better situated to detect such disease representative occurrences, given that a subject-specific sample analysis may account for discrepancies that are normal to the subject, even if they are not normal for a broader population. It will be appreciated that, in some instances, a population-level normal data set may nonetheless provide advantages, such as providing higher accuracy as to the probability of a given discrepancy occurring as a result of a sequencing error due to a high number of reads aligned to a region (e.g., including reads generated from multiple samples and/or multiple subjects). It will also be appreciated that, in some instances, accessing population-level methylation and subject-specific methylation data may provide advantages over methods that individually access population-level methylation data or subject-specific methylation data.

### V. Exemplary Probe Capture Protocol

Some disclosures indicate how particular bases and/or methylations may be informative as to whether a given sequence read corresponds to a disease, which may be used to indicate (for example) whether a subject has a given disease, a stage of a disease of the subject, a progression of the disease, an efficacy of a treatment for the subject, etc. Thus, in some instances, performing a targeted enrichment for a subject may be particularly informative, as this approach may amplify signals from a given disease (or suspected disease). Additionally, or alternatively, developing and/or using a probe that detects whether the particular bases and/or methylations may be particularly informative.

Certain embodiments may include one or more labels. The one or more labels may be attached to one or more capture probes, nucleic acid molecules, beads, primers, or a combination thereof. Examples of labels include, but are not limited to, detectable labels, such as radioisotopes, fluorophores, chemiluminophores, chromophore, lumiphore, enzymes, colloidal particles, and fluorescent microparticles, quantum dots, as well as antigens, antibodies, haptens, avidin/streptavidin, biotin, haptens, enzymes cofactors/substrates, one or more members of a quenching system, a chromogens, haptens, a magnetic particles, materials exhibiting nonlinear optics, semiconductor nanocrystals, metal nanoparticles, enzymes, aptamers, and one or more members of a binding pair.

Certain embodiments may include one or more capture probes, a plurality of capture probes, or one or more capture probe sets. In some instances, the one or more capture probes, the plurality of capture probes, or the one or more capture probe sets may comprise one or more self-identifying capture probes, a plurality of self-identifying capture probes, or one or more self-identifying capture probe sets, as described herein. Typically, the capture probe comprises a nucleic acid binding site. The capture probe may further comprise one or more linkers. The capture probes may further comprise one or more labels. The one or more linkers may attach the one or more labels to the nucleic acid binding site. In some embodiments, the one or more capture probes, the plurality of capture probes, or the one or more capture probe sets may further comprise one or more normalization probes, a plurality of normalization probes, or one or more normalization probe sets.

Capture probes may hybridize to one or more nucleic acid molecules in a sample. Capture probes may hybridize to one or more genomic regions. Capture probes may hybridize to one or more genomic regions within, around, near, or spanning one or more genes, exons, introns, UTRs, or a combination thereof. Capture probes may hybridize to one or more genomic regions spanning one or more genes, exons, introns, UTRs, or a combination thereof. Capture probes may hybridize to one or more known inDels. Capture probes may hybridize to one or more known structural variants.

Certain embodiments may include 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, 1000 or more, 1200 or more, 1500 or more, 1800 or more, 2000 or more, 2500 or more, or 3000 or more capture probes or capture probe sets. The one or more capture probes or capture probe sets may be different, similar, identical, or a combination thereof.

The one or more capture probe may comprise a nucleic acid binding site that hybridizes to at least a portion of the one or more nucleic acid molecules or variant or derivative thereof in the sample or subset of nucleic acid molecules. The capture probes may comprise a nucleic acid binding site that hybridizes to one or more genomic regions. The capture probes may hybridize to different, similar, and/or identical genomic regions. The one or more capture probes may be at least about 50%, 55%, 60%, 65%, 70%, 75%, 80%, 85%, 90%, 95%, 97%, 99% or more complementary to the one or more nucleic acid molecules or variant or derivative thereof.

The capture probes may comprise one or more nucleotides. The capture probes may comprise 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more nucleotides. The capture probes may comprise about 100 nucleotides. The capture probes may comprise between about 10 to about 500 nucleotides, between about 20 to about 450 nucleotides, between about 30 to about 400 nucleotides, between about 40 to about 350 nucleotides, between about 50 to about 300 nucleotides, between about 60 to about 250 nucleotides, between about 70 to about 200 nucleotides, or between about 80 to about 150 nucleotides. In some aspects of the disclosure, the capture probes comprise between about 80 nucleotides to about 100 nucleotides.

### VI. Exemplary Techniques for Targeted Enrichment of a Biological Sample

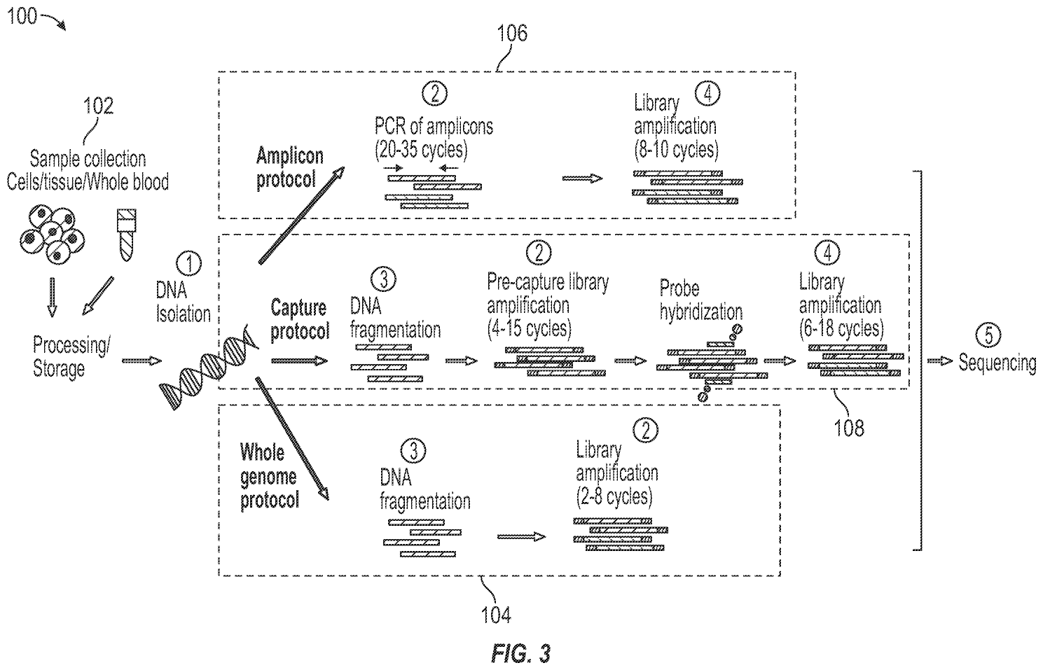

FIG. 3 shows a schematic diagram illustrating a process 100 for targeted enrichment of a biological sample 102, according to some embodiments. The biological sample 102 can include any tissue (or bodily fluid) derived from a subject. In some instances, the biological sample is a cell-free sample, which may include a mixture of nucleic acid molecules from the subject and potentially nucleic acid molecules from pathogens (e.g., virus, tumor cells). The biological sample can include bodily fluid, such as blood, plasma, serum, urine, or other fluid from different parts of the body (e.g., thyroid or breast) of the subject.

In the past, sequencing nucleic acid molecules of the biological sample 102 was tedious and time consuming. The recent development of next-generation sequencing (NGS) techniques, however, have allowed generation of large volumes of sequencing data in shorter amount of time. The NGS techniques significantly decreased the amount of time needed for analyzing samples of a subject (e.g., the biological sample 102) and have allowed comprehensive analyses. For example, a whole-genome sequencing (WGS) technique 104 can be used to determine the entirety, or nearly the entirety, of the nucleic acid sequence of a subject's genome at a single time. To further facilitate the analysis, the WGS technique 104 can also include amplifying the nucleic acid molecules of the sample during the library preparation step. Despite the increase in efficiency, analysis of whole-genome sequencing data spanning an entire genome can be time-consuming and may take weeks to process.

To further increase efficiency of nucleic acid analysis, various targeted enrichment strategies can be implemented. For example, a polymerase chain reaction (PCR) technique 106 have often been used for the clinical diagnosis of infectious diseases, in which the PCR technique 106 can include amplifying short and conserved genomic regions to produce a set of amplicons prior to the library preparation step. The set of amplicons can be sequenced to provide information on the presence/absence or relative abundance of target DNA or RNA (e.g., viral DNA or RNA, tumor DNA or RNA). The PCR technique 106 has numerous advantages, such as low cost, rapid processing and results acquisition, automation, sensitivity and specificity. Relative to the WGS technique 104, the PCR technique 106 can provide partial information on the genetic diversity, genotype, functional potential, and nutritional requirements as well as virulence or antibiotic-resistance.

The targeted enrichment strategy can also include hybridization-based capture technique 108. The hybridization-based capture technique 108 can be applied directly applied after nucleic acid extraction and library preparation of the biological sample 102. In particular, fragmented shotgun libraries of the biological sample 102 can be denatured by heating, and the denatured fragments can be subjected to hybridization with DNA or RNA single-stranded oligonucleotides (called also ‘probes’ or ‘baits’) specific to target genomic regions. The hybridization-based capture technique 108 can be advantageous for genotyping and rare genetic variant detection. This is because the hybridization-based capture technique 108 does not require PCR primer design, and it is thus less likely to miss mutations and performs better with respect to sequence complexity.

### VII. Exemplary Process for Assigning a Probe-set Identifier to a Probe Set

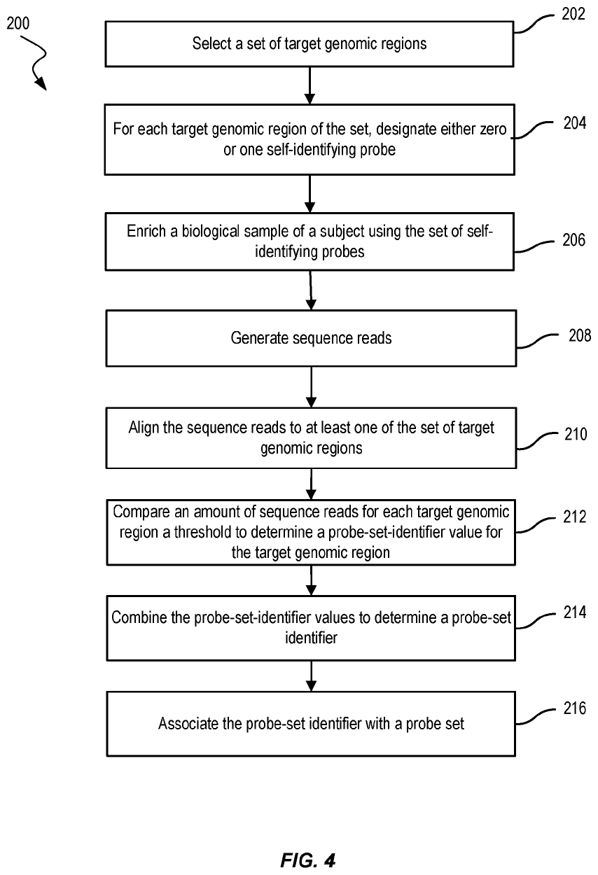

FIG. 4 includes a flowchart 200 illustrating an example of a method of assigning a probe-set identifier of a corresponding probe set, according to some embodiments. Some of the operations described in flowchart 200 may be performed by, for example, a computer system that can analyze sequence reads corresponding to an enriched biological sample. Although flowchart 200 may describe the operations as a sequential process, in various embodiments, many of the operations may be performed in parallel or concurrently. In addition, the order of the operations may be rearranged. An operation may have additional steps not shown in the figure.

Furthermore, some embodiments of the method may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

At step 202, a set of target genomic regions are selected. In some instances, the set of target genomic regions are selected based on one or more genomic features (such as, for example, the presence of polymorphism(s), methylation status, etc.). Sequencing data corresponding to each of the target genomic regions can be used to derive a corresponding probe-set-identifier value. In some instances, the set of target genomic regions are selected from at least a portion of a human reference genome.

In some instances, a certain portion of a genome is set aside and used only for determining the probe-set identifier. Thus, any sequencing data which aligns to these target genomic regions can be interpreted only for determining the probe-set identifier. The target genomic regions can be from a continuous genomic region, but it can also correspond to a plurality of discontinuous genomic regions spread across one or more chromosomes. In some instances, the discontinuous genomic regions can be desirable for a number of reasons, including robustness over sample-to-sample variation. Additional aspects of identifying target genomic regions are described below.

At step 204, for each target genomic region of the set, either zero or one self-identifying probe can be designated. Thus, when the particular probe set is used to enrich the sample, sequencing data generated from the enriched sample can indicate that a target genomic region assigned with the capture probe may result in a larger amount of sequence reads relative to those of other target genomic regions that were not assigned with a respective capture probe. The designated self-identifying probes can be assigned as a set of self-identifying probes for generating a corresponding probe-set identifier of a probe set.

At step 206, a biological sample of a subject is enriched for nucleic acid molecules targeted by the set of self-identifying probes.

The enrichment can include using hybridization-based capture technique (e.g., the hybridization-based capture technique 108 of FIG. 3), in which the set of self-identifying probes are applied after nucleic acid extraction and library preparation of the biological sample. In particular, fragmented shotgun libraries of the biological sample 102 be denatured by heating, and the denatured fragments can be subjected to hybridization with DNA or RNA single-stranded oligonucleotides (called also ‘probes’ or ‘baits’) specific to target genomic regions.

At step 208, the enriched sample is sequenced to generate sequence reads. A sequence read may be obtained using various techniques, including performing an NGS sequencing technique, a sequencing-by-synthesis technique, or performing single molecule sequencing, and performing nanopore sequencing. As part of an analysis of a biological sample, at least 1,000 sequence reads can be analyzed. As other examples, at least 10,000 or 50,000 or 100,000 or 500,000 or 1,000,000 or 5,000,000 sequence reads, or more, can be analyzed.

At step 210, the sequence reads are aligned to at least one of the target genomic regions. The aligned sequence reads can be used to identify a sequencing coverage for each of the target genomic regions.

At step 212, an amount of sequence reads for each target genomic region can be compared to a threshold to determine a probe-set-identifier value for the target genomic region. If the amount of sequence reads exceeds the threshold, then the corresponding target genomic region can be encoded as a “1.” Otherwise, the corresponding target genomic region can be encoded as a “0.”

At step 214, the probe-set-identifier value for each target genomic region is combined into a probe-set identifier. For example, the probe-set-identifier values corresponding to the set of target genomic regions can be combined together to determine the probe-set identifier. In some instances, the probe-set identifier is a N-bit binary value that can be interpreted as a number, date, text or other form of the probe-set identifier, in which N represents a number of target genomic regions in the set. In some instances, the encoding of the probe-set identifiers involves values other than binary numbers, such as hexadecimal or decimal numbers. In such cases, multiple thresholds for encoding the probe-set-identifier value can be used.

At step 216, the probe-set identifier is associated with the probe set. As described above, the probe-set identifier can be used to identify the probe set without accessing any external resources. The probe-set identifier can be used to generate a result indicating that the probe set includes one or more subject-specific capture probes that enrich the biological sample for a set of nucleic acid molecules of the plurality of nucleic acid molecules. The set of nucleic acid molecules facilitate a determination of a classification of pathology for the subject.

### VIII. Determining a Probe-set Identifier of a Probe Set

As described herein, the present techniques can include using a probe set that includes a set of self-identifying probes for determining a corresponding probe-set identifier. The set of self-identifying probes can be designed (e.g., using the process 200 of FIG. 4) to capture nucleic acid molecules from specific parts of the human genome, and the set of self-identifying probes are different from self-identifying probes of other probe sets A sequencing coverage derived from the set of self-identifying probes can be interpreted into a probe-set identifier for identifying a corresponding probe set, which can be performed without having to refer to any design information database. For example, the nucleic acid sequencing coverage of the set of self-identifying probes can be interpreted as “probe set #43,207,” and one can confirm whether the corresponding probe set was an expected probe set for the subject. If the probe set was not the expected set, the probe-set identifier may be used as a guidepost to determine why the incorrect probe set was identified and to track down the expected probe set. In some instances, the probe-set identifier includes a number, or text, a date on which the probe set was designed, other related information (e.g., an identifier of the subject), or a combination of those.

### A. Exemplary Implementation

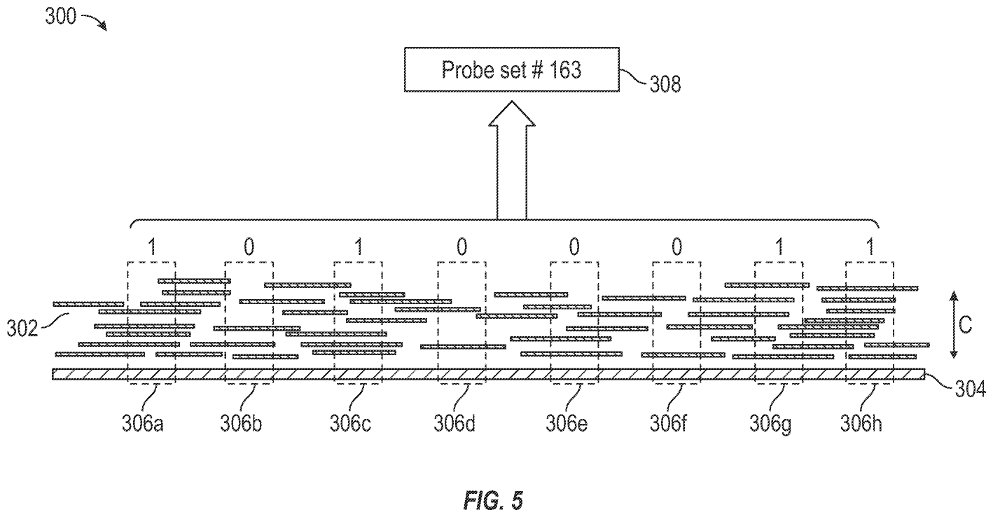

FIG. 5 shows an example of a schematic diagram 300 for determining a probe-set identifier of a probe set, according to some embodiments. A plurality of sequence reads 302 can be obtained from a biological sample (e.g., the biological sample 102 of FIG. 3), in which the biological sample is enriched with the probe set.

To illustrate, nucleic acid molecules of a biological sample derived from the blood plasma of a subject (e.g., a subject with pathology) can be obtained. The nucleic acid molecules are randomly sheared into smaller nucleic acid fragments. In some instances, the median length of the nucleic acid fragments can be in the range of 140-400 bases. The nucleic acid fragment can then be converted into sequencing libraries.

A probe set (e.g., a hybridization-based capture probe set) can then be applied to the sequencing libraries to enrich nucleic acid molecules that correspond to genomic regions targeted by the set of self-identifying probes of the probe set. The probe set can be created using the Agilent SureSelect system, the Twist custom capture probe set platform, or other systems. Additionally, or alternatively, each probe of the probe set can be individually synthesized on a DNA or RNA synthesizing instrument, and the synthesized probes can be pooled together into the probe set. Each probe can be 60-150 bases long and may be comprised of DNA, RNA or other form of nucleic acid sequence. After the biological sample is enriched, sequencing can be performed to generate sequencing data for the biological sample. For example, DNA sequencing using 2×150 paired-end reads from an Illumina NovaSeq-6000 instrument, can be performed on the enriched biological sample.

The sequencing data can then be mapped to one of reference sequences (e.g., GRCh37 or GRCh38). The mapped sequencing data can be used to identify sequencing coverage related to each target genomic region, and the sequencing coverages can be used to determine values of the probe set identifier. In some instances, the sequencing coverage is determined by counting a number of sequence reads which map to each of a target genomic region or counting a number of sequence reads that cover a specific position within each target genomic region, or other suitable metrics.

Each of the plurality of sequence reads 302 can be aligned to a corresponding portion of a reference sequence 304. In some instances, the reference sequence 304 represent at least part of a human reference genome. After alignment, a set of target genomic regions 306a-h can be selected. As described above, one or more of the self-identifying probes of the probe set can enrich the biological sample for nucleic acid molecules that align to a corresponding target genomic region (e.g., the target genomic region 306a). Such configuration of the self-identifying probes can facilitate the encoding of the probe-set identifier.

A sequencing coverage for each of the target genomic regions 306a-h can be determined, and such sequencing coverage is compared through a threshold value to determine a value of the probe-set identifiers. In some instances, the value includes a binary value of “0” or “1.” In this example, each of the target genomic regions 306a-h can represent either a binary value of “0” and “1.” The sequence of binary values can encode an 8-bit binary number that represents a probe-set identifier 308. In this example, the 8-bit binary number “10100011” can be converted into a decimal number “163,” and the decimal number “163” can be the probe-set identifier of the probe set.

### B. Process for Determining a Probe-set Identifier of a Probe Set

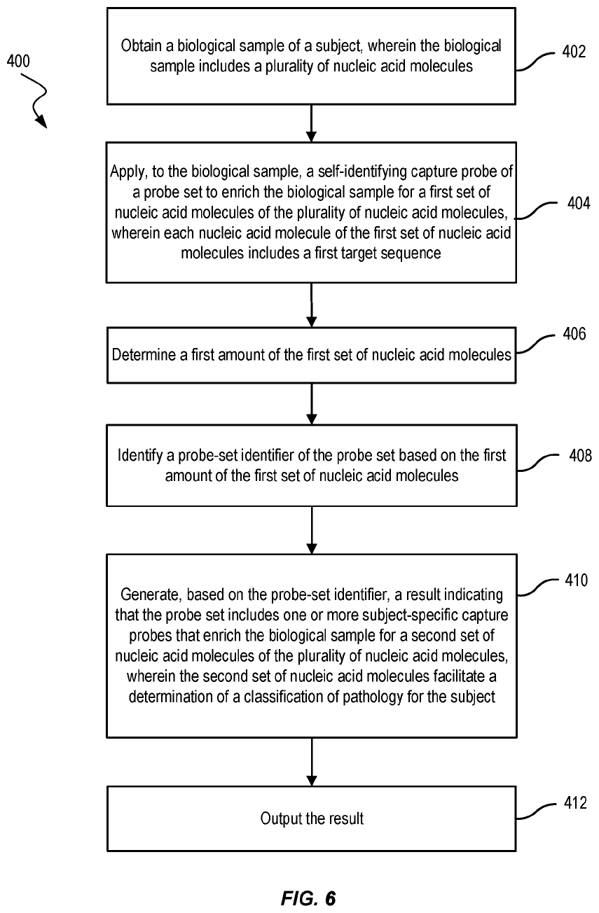

FIG. 6 includes a flowchart 400 illustrating an example of a method of determining a probe-set identifier of a corresponding probe set, according to some embodiments. Some of the operations described in flowchart 400 may be performed by, for example, a computer system that can analyze sequence reads corresponding to an enriched biological sample. Although flowchart 400 may describe the operations as a sequential process, in various embodiments, many of the operations may be performed in parallel or concurrently. In addition, the order of the operations may be rearranged. An operation may have additional steps not shown in the figure. Furthermore, some embodiments of the method may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

At operation 402, a biological sample of a subject can be obtained. The biological sample can include a plurality of nucleic acid molecules. In some instances, the biological sample includes nucleic acid derived from tumor or healthy cells. The biological sample can include a plurality of nucleic acid molecules. The nucleic acid molecules may include DNA or RNA. In some instances, the biological sample includes cell-free nucleic acid molecules, including circulating tumor DNA (ctDNA) or circulating tumor RNA (ctRNA). Additionally, or alternatively, the biological sample may include a tissue sample or a core needle biopsy sample, in which the nucleic acid molecules can be obtained from circulating tumor cells in the sample.

At operation 404, a self-identifying capture probe of a probe set can be applied to enrich the biological sample for a first set of nucleic acid molecules of the plurality of nucleic acid molecules. In some instances, the self-identifying capture probe and other capture probes of the probe set are applied together to enrich the biological sample. Each nucleic acid molecule of the first set of nucleic acid molecules includes a first target sequence. The first target sequence can correspond to a sequence targeted by the self-identifying capture probe.

At operation 406, a first amount of the first set of nucleic acid molecules can be determined. In some instances, the first amount of the first set of nucleic acid molecules can be determined by: sequencing the plurality of nucleic acid molecules of the enriched biological sample to obtain a plurality of sequence reads; aligning each of the plurality of sequence reads to a corresponding portion of a human reference genome; identifying, from the aligned sequence reads, a set of sequence reads that correspond to the first target sequence; determining an amount of the set of sequence reads; and identifying, based on the amount of the set of sequence reads, a sequencing coverage for the probe set.

At operation 408, a probe-set identifier of the probe set can then be identified based on the first amount of the first set of nucleic acid molecules. In some instances, the probe-set identifier is identified based on determining whether the sequencing coverage exceeds a predetermined threshold. If the sequencing coverage exceeds the predetermined threshold, a first value of the probe-set identifier can be determined, in which the first value is predictive of a presence of the first target sequence in the biological sample. In contrast, if the sequencing coverage does not exceed the predetermined threshold, a second value of the probe-set identifier can be determined, in which the second value is predictive of an absence of the first target sequence in the biological sample.

At operation 410, a result is generated based on the probe-set identifier. The result indicates that the probe set includes one or more subject-specific capture probes that enrich the biological sample for a second set of nucleic acid molecules of the plurality of nucleic acid molecules. The second set of nucleic acid molecules facilitate a determination of a classification of pathology for the subject. The probe set that includes the one or more subject-specific capture probes can thus be correctly selected and used for identifying and tracking genetic mutations in the corresponding subject.

At operation 412, the result is outputted. In some instances, the second set of nucleic acid molecules are obtained from the biological sample enriched with subject-specific capture probes of the probe set. The second set of nucleic acid molecules can be sequenced and aligned to a reference sequence to identify and track genetic mutations associated with the subject. The identified genetic mutations can be used to determine the classification of pathology for the subject. Process 400 terminates thereafter.

### IX. Determining Probe-set Identifier Values Based on Sequencing Coverage

Sequence reads that align to a genomic region targeted by a self-identifying probe can be used to determine sequencing coverage. To determine a value that encodes the probe-set identifier, a distribution of sequencing coverage across the target genomic region can be determined. Then, a peak within the distribution of sequencing coverage can be used to determine the value that encodes the probe-set identifiers. The peak can indicate a location within the target genomic region to which the largest amount of sequence reads is aligned. In some instances, the peak is approximately centered in the target genomic region of the capture probe, with the width of the coverage peak being 100-500 bases.

A metric of the sequencing coverage can be determined based on the peak of a corresponding target genomic region. In some instances, each of the capture probes used for this purpose is designed to target the center of a 1,000-base target genomic region. For example, if 32 target genomic regions are used, the probe-set identifier can be encoded into a 32-bit binary code, allowing unique probe-set identifiers to be created up to 2³² (over 4 billion) probe sets. With respect to a size of the target genomic regions, 32-bit probe-set identifiers can require setting aside 32,000 bases (i.e., ˜0.001% of the genome).

As described herein, binary information can be encoded by the metric by comparing the metric to a predetermined threshold value. The comparison between the metric and the threshold indicates whether the peak for a particular target genomic region should represent a probe-set-identifier value. Additionally, or alternatively, other techniques can be used to derive a code from the nucleic acid sequencing coverage. For example, a capture probe targeting a target genomic region can be used, in which the target genomic region includes a first and second genomic sub-regions. In this example, a value of “1” can be encoded if a peak of the sequencing coverage is centered on the first genomic sub-region (e.g., the right half of the target genomic region), and a value of “0” can be encoded if the peak of the sequencing coverage is centered in the second genomic sub-region (e.g., the left half of the target genomic region). To further illustrate, if a target genomic region was 1,000 bases long, the result would be a “1” if the sequencing coverage peak was at a position within the target genomic region of 501-1,000 and a “0” if the sequencing coverage peak was at position 1-500 in the target genomic region. In either case, a single coverage peak would be detected in each of the set of target genomic regions.

Continuing with the above example, if no sequencing coverage peak was detected above threshold in the entire 1,000-base target genomic region, the probe-set-identifier value (e.g., “0,” “1”) may be encoded but instead determined that the probe-set identifier process did not operate properly. Thus, failure to detect a peak in the target genomic region can be distinguished as an assay failure in that genomic region, not a confident detection of a “0” value. Similarly, if a nucleic acid sequencing coverage peak is detected above threshold in both the 1-500 range and the 501-1,000 range, it can also indicate assay malfunction, not a confident detection of “1” value.

### X. Identifying Target Genomic Regions for Determining Values of the Probe-set Identifier

In some embodiments of the present disclosure provide a technical advantage over conventional techniques by using self-identifying probes to determine whether a probe set used on a nucleic acid sample of a subject is in fact the expected sample. Because coverage of sequence data targeted by the self-identifying probes can be used to determine a corresponding probe-set identifier, the present techniques can accurately identify the probe set even when external events (e.g., accidental mix-ups with other probe sets) cause other identification resources to become ineffective. Further, the self-identifying probes can enrich nucleic acid molecules corresponding to target genomic regions for encoding the probe-set identifier, such that small genetic variations (e.g., single-nucleotide polymorphisms) in some of the target genomic regions do not alter the result. Therefore, the present techniques facilitate accurate and reliable self-identification of probe sets, without requiring databases to retrieve the corresponding database records.

To identify a corresponding probe set while remaining independent from testing of the subject whose DNA or RNA is analyzed, the set of self-identifying probes would not simply target genomic regions in which genetic variants of the subject are found. Rather, the set of self-identifying probes may correspond to target genomic regions at which nucleic acid sequence data was captured, regardless of whether the target genomic regions include any genetic variants.

In some instances, a hybridization-based capture technique is used to enrich the sample of the subject for nucleic acid molecules corresponding to a set of target genomic regions. Such targeted enrichment can facilitate generation of the output (e.g., the probe-set identifier) regardless of whether the sample includes small variants in part of the target genomic regions. For example, if there is a capture probe for enriching nucleic acid molecules corresponding to a genomic location at location “X,” then the derived nucleic acid sequence data can be expected at or nearby the location X regardless of whether there is a single-nucleotide polymorphism (SNP) or other genetic variants. On the other hand, if there is no capture probe for location X (or any nearby location), then little or no nucleic acid sequence data would be expected at that location. Thus, the presence or absence of sequence data at a particular location provides information about whether a probe in the probe set is present for that location.

### A. Determining a Threshold Using Normalization Probes

In some instances, using hybridization-based capture probe sets results in sequencing coverage that differs from the expected coverage. The result can be due to genetic variation in the sample. The result can also be due to varying laboratory conditions, including variations in time allowed for hybrid capture, temperature at which the hybridization is conducted, amplification before or after capture, and combination of various assays performed on a single flow cell. To anticipate such discrepancies and improve the ability to discriminate the sequencing depth as a “1” or “0,” the capture probe set can be configured to include one or more normalization probes, which can be independent of the corresponding probe-set identifier. The nucleic acid sequencing coverage detected in a genomic region targeted by normalization probes can be used to normalize the threshold used for determining a relative amount of sequence reads targeted by capture probes for encoding the probe-set identifier. In some instances, the probe set can include a plurality of normalization probes. If there are multiple normalization probes, various normalizing schemes can be used for determining the threshold. For example, each of the plurality of normalization probes can be used to identify a particular threshold for determining a probe-set-identifier value for a corresponding target genomic region. In another example, the plurality of normalization probes can be used together to identify the particular threshold for determining a probe-set-identifier value for each of the target genomic regions.

### B. Using Redundant Target Genomic Regions for Determining the Probe-Set Identifier

As described above, an assay performed on a target genomic region may fail to provide a definitive “1” or “0” code. This may be due to a variety of reasons, including failed probe synthesis, a deletion in a genome of the sample which overlaps the target genomic region, or by other mechanisms. A self-identifying probe set design can be made more robust by allocating more than one genomic region for each bit being encoded. For example, three separate genomic regions can be used, perhaps on three separate chromosomes, to encode each bit. If the assay fails in one or two of these genomic regions, the result from the third targeted genomic region can still be used to determine the bit. If the assay succeeds in all three target genomic regions, but the results differ (e.g., two genomic regions yield “1” and one genomic region yields “0”), then a voting scheme can be implemented to report the most frequently appearing result. The above redundancy can be used to improve the robustness of determination of each bit of the probe-set identifier, in turn resulting in the robustness of detection of the probe set.

In some instances, there is a probability that a target genomic region (or a set of target genomic regions as described above) results in an incorrect binary code. In such cases, the errors can be detected and, in some cases, even corrected by using a parity bit or an error correcting code.

### C. Selecting Non-Desirable Genomic Regions as Target Genomic Regions

In some instances, it is undesirable to set aside a part of a genome of subject for determining the probe-set identifier. For example, certain genomic regions of a genome can be used for other purposes of the personalized assay. To address these challenges, self-identifying probes of the probe set can target portions of the genome that are considered undesirable for the other uses.

For example, for a probe set designed to detect minimal residual disease (MRD), the probe set is typically configured to search for somatic variants identified in the subject's tumor. In this example, the probe set is typically configured to avoid undesirable genomic regions. This may include genomic regions with degenerate mapping, including the regions that are affected by a pseudo-gene or tandem duplication (for example). Similarly, undesirable genomic regions also can include those of the reference sequence that are referred to as “compressions” (see, e.g., Dewey, et. al, Phased Whole-Genome Genetic Risk in a Family Quartet Using a Major Allele Reference Sequence, PLOS Genetics, vol. 7, issue 9, 2011), in which the actual physical genome has a duplication, but the reference sequence only reflects one copy. The probe set is thus configured to avoid the above undesirable genomic regions which can result in inaccurate and suboptimal sequence data.

However, while the above-referenced genomic regions may be less optimal for sensitive detection of somatic variants, such genomic regions can be targeted by the self-identifying probes of the probe set. In this manner, using these genomic regions would be less likely to interfere with other uses of the probe set. In another example, the genomic regions targeted by the self-identifying probes can correspond to genomic regions with no known function, including intergenic regions or certain portions of long introns.

In yet another example, the target genomic regions of the self-identifying probes can include genomic regions of the mitochondrial chromosome. Typically, the mitochondrial chromosome is not frequently used for other applications of custom assays, because mitochondrial DNA includes several copies that include small variants. The reasons which make the mitochondrial chromosome undesirable for those other applications of custom assays may not impact the use for self-identifying probes. Thus, portions of the mitochondrial chromosome can be considered as candidate for genomic regions to be targeted by the self-identifying probes of the probe set.

### D. Using Non-Human DNA or RNA as Target Genomic Regions

In some instances, non-human DNA or RNA is spiked into the biological sample, and genomic regions corresponding to the non-human DNA or RNA can be targeted by the self-identifying probes of the probe set. In effect, there is no longer a need to set aside a portion of the human genome to determine the probe-set identifier of the probe set. The non-human DNA or RNA can be derived from a naturally occurring sample (e.g., from a non-human species). In some instances, the non-human DNA or RNA are completely synthetic sequences. Thus, if self-identifying probes targeting such non-human nucleic acid sequences are used on a biological sample with only human DNA or RNA, not many sequence reads (if any) can be expected from the target genomic regions. On the other hand, if each biological sample of a human subject is combined with an amount of non-human DNA or RNA, then sequences from the non-human DNA could be captured by the self-identifying probes to subsequently allow determination of the probe-set identifier. In some instances, the non-human DNA is derived from viral DNA “Phi-X,” which is generally used for quality control of sequencing data. The non-human DNA or RNA can represent a very small portion of the total nucleic acid sequence data (e.g., 1%), but can be sufficient enough for implementing the self-identification methods described herein.

### E. Intermixed Genomic Regions for Determining the Probe-set Identifier

In some instances, genomic regions targeted by the self-identifying probes are intermixed with the regions targeted by other capture probes. As a result, no part of the human genome needs to be set aside for self-identification. The capture probes of the probe set can thus be used as pairs or groups that target genomic regions that are either closely spaced or widely spaced. Such configuration can be feasible as many applications of custom assays selectively capture only a very small portion of the human genome. For example, a custom assay with 500 probes, each targeting 120 bases, would cover only 60,000 bases (0.002%) of the human genome. Thus, even if the target genomic regions used for determining the probe-set identifier were not segregated from the other uses, the overlap between these genomic regions may still be very low. In the event of a possible overlap, such few interactions can be rare enough that they could be addressed using the redundant target genomic regions and/or the error-correcting codes.

In some instances, the self-identifying probes are implemented in pairs or other small groups. As a result, the sequencing coverage from the self-identifying probes can be distinguished from probes used for other purposes, because the pairs of self-identifying probes can generate a signature “double-peak” on the sequencing coverage plot. In some instances, these grouped peaks of sequencing coverages are even more clearly distinguished from sequencing coverages of other probes if the target genomic regions are located far apart from each other on the genome (e.g., separate chromosomes).

### F. Encoding Multiple Values for Each Target Genomic Region

In some instances, a genomic region targeted by the self-identifying probe provides an increased amount of information, so as to reduce the number of probes needed to encode the probe-set identifier. Depending on the amount of the information to be encoded, a number of self-identifying probes may become prohibitive if a single bit (“1” vs “0”) is captured by each self-identifying capture probe. As such, additional information can be encoded in each self-identifying capture probe based on the corresponding nucleic acid sequencing coverage peaks. For example, a self-identifying capture probe can be configured to produce a nucleic acid sequencing coverage peak that includes: (i) 250 bases full-width at half-maximum (FWHM); and (ii) a center position of the peak having a precision of greater than 100 bases. Using this configuration for each nucleic acid sequencing coverage peak, 256 different positions (corresponding to 8 bits of information since 2\hat{ }8=256) can be encoded for each self-identifying probe, if a genomic region targeted by the self-identifying probe includes 256×100=25,600 bases that are available for encoding the probe-set identifier.

Using the above example technique, four capture probes can together encode a 32-bit probe-set identifier. Although such technique may require setting aside a larger portion of the genome, e.g., 25,600×4=102,400 bases, the larger portion can still be a very small part (e.g., 0.004%) of the genome. In some instances, multiple capture probes sparsely populate a shared genomic region, such that the sequencing coverage peaks do not overlap or can be easily separated.

### XI. Assays and Amplification Techniques

Certain embodiments may include conducting one or more assays on a sample comprising one or more nucleic acid molecules. Producing two or more subsets of nucleic acid molecules may comprise conducting one or more assays. The assays may be conducted on a subset of nucleic acid molecules from the sample. The assays maybe conducted on one or more nucleic acids molecules from the sample. The assays may be conducted on at least a portion of a subset of nucleic acid molecules. The assays may comprise one or more techniques, reagents, capture probes, primers, labels, and/or components for the detection, quantification, and/or analysis of one or more nucleic acid molecules.

It will be appreciated that a given assay may be performed to facilitate identifying whether there are any variants in a sequence of a subject, to predict which variant(s) exist in a sequence of a subject, and/or a methylation percentage at one or more positions for a subject. Thus, a given assay may be used (for example) only to identify bases and/or variants for a subject but not to inform a prediction of a methylation state or methylation percentage (or the reverse).

Assays may include, but are not limited to, sequencing, amplification, hybridization, enrichment, isolation, elution, fragmentation, detection, quantification of one or more nucleic acid molecules. Assays may include methods for preparing one or more nucleic acid molecules.

Certain embodiments may include conducting one or more amplification reactions on one or more nucleic acid molecules in a sample. The term “amplification” refers to any process of producing at least one copy of a nucleic acid molecule. The terms “amplicons” and “amplified nucleic acid molecule” refer to a copy of a nucleic acid molecule and can be used interchangeably. The amplification reactions can comprise PCR-based methods, non-PCR based methods, or a combination thereof. Examples of non-PCR based methods include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. PCR-based methods may include, but are not limited to, PCR, HD-PCR, Next Gen PCR, digital RTA, or any combination thereof. Additional PCR methods include, but are not limited to, linear amplification, allele-specific PCR, Alu PCR, assembly PCR, asymmetric PCR, droplet PCR, emulsion PCR, helicase dependent amplification HDA, hot start PCR, inverse PCR, linear-after-the-exponential (LATE)-PCR, long PCR, multiplex PCR, nested PCR, hemi-nested PCR, quantitative PCR, RT-PCR, real time PCR, single cell PCR, and touchdown PCR.

Certain embodiments may include conducting one or more hybridization reactions on one or more nucleic acid molecules in a sample. The hybridization reactions may comprise the hybridization of one or more capture probes to one or more nucleic acid molecules in a sample or subset of nucleic acid molecules. The hybridization reactions may comprise the hybridization of one or more self-identifying probes to one or more nucleic acid molecules in a sample or subset of nucleic acid molecules. The hybridization reactions may comprise hybridizing one or more capture probe sets to one or more nucleic acid molecules in a sample or subset of nucleic acid molecules. The hybridization reactions may comprise hybridizing one or more self-identifying probe sets to one or more nucleic acid molecules in a sample or subset of nucleic acid molecules. The hybridization reactions may comprise one or more hybridization arrays, multiplex hybridization reactions, hybridization chain reactions, isothermal hybridization reactions, nucleic acid hybridization reactions, or a combination thereof. The one or more hybridization arrays may comprise hybridization array genotyping, hybridization array proportional sensing, DNA hybridization arrays, macroarrays, microarrays, high-density oligonucleotide arrays, genomic hybridization arrays, comparative hybridization arrays, or a combination thereof. The hybridization reaction may comprise one or more capture probes, one or more beads, one or more labels, one or more subsets of nucleic acid molecules, one or more nucleic acid samples, one or more reagents, one or more wash buffers, one or more elution buffers, one or more hybridization buffers, one or more hybridization chambers, one or more incubators, one or more separators, or a combination thereof.

Certain embodiments may include conducting one or more enrichment reactions on one or more nucleic acid molecules in a sample. The enrichment reactions may comprise contacting a sample with one or more beads or bead sets. The enrichment reaction may comprise differential amplification of two or more subsets of nucleic acid molecules based on one or more genomic region features. For example, the enrichment reaction comprises differential amplification of two or more subsets of nucleic acid molecules based on GC content. Alternatively, or additionally, the enrichment reaction comprises differential amplification of two or more subsets of nucleic acid molecules based on methylation state. The enrichment reactions may comprise one or more hybridization reactions. The enrichment reactions may further comprise isolation and/or purification of one or more hybridized nucleic acid molecules, one or more bead bound nucleic acid molecules, one or more free nucleic acid molecules (e.g., capture probe free nucleic acid molecules, or bead free nucleic acid molecules), one or more labeled nucleic acid molecules, one or more non-labeled nucleic acid molecules, one or more amplicons, one or more non-amplified nucleic acid molecules, or a combination thereof. Alternatively, or additionally, the enrichment reaction may comprise enriching for one or more cell types in the sample. The one or more cell types may be enriched by flow cytometry.

The one or more enrichment reactions may produce one or more enriched nucleic acid molecules. The enriched nucleic acid molecules may comprise a nucleic acid molecule or variant or derivative thereof. For example, the enriched nucleic acid molecules comprise one or more hybridized nucleic acid molecules, one or more bead bound nucleic acid molecules, one or more free nucleic acid molecules (e.g., capture probe free nucleic acid molecules, or bead free nucleic acid molecules), one or more labeled nucleic acid molecules, one or more non-labeled nucleic acid molecules, one or more amplicons, one or more non-amplified nucleic acid molecules, or a combination thereof. The enriched nucleic acid molecules may be differentiated from non-enriched nucleic acid molecules by GC content, molecular size, genomic regions, genomic region features, or a combination thereof. The enriched nucleic acid molecules may be derived from one or more assays, supernatants, eluents, or a combination thereof. The enriched nucleic acid molecules may differ from the non-enriched nucleic acid molecules by mean size, mean GC content, genomic regions, or a combination thereof.

Certain embodiments may include conducting one or more isolation or purification reactions on one or more nucleic acid molecules in a sample. The isolation or purification reactions may comprise contacting a sample with one or more beads or bead sets. The isolation or purification reaction may comprise one or more hybridization reactions, enrichment reactions, amplification reactions, sequencing reactions, or a combination thereof. The isolation or purification reaction may comprise the use of one or more separators. The one or more separators may comprise a magnetic separator. The isolation or purification reaction may comprise separating bead bound nucleic acid molecules from bead free nucleic acid molecules. The isolation or purification reaction may comprise separating capture probe hybridized nucleic acid molecules from capture probe free nucleic acid molecules. The isolation or purification reaction may comprise separating a first subset of nucleic acid molecules from a second subset of nucleic acid molecules, wherein the first subset of nucleic acid molecules differ from the second subset on nucleic acid molecules by mean size, mean GC content, genomic regions, or a combination thereof.

Certain embodiments may include conducting one or more elution reactions on one or more nucleic acid molecules in a sample. The elution reactions may comprise contacting a sample with one or more beads or bead sets. The elution reaction may comprise separating bead bound nucleic acid molecules from bead free nucleic acid molecules. The elution reaction may comprise separating capture probe hybridized nucleic acid molecules from capture probe free nucleic acid molecules. The elution reaction may comprise separating a first subset of nucleic acid molecules from a second subset of nucleic acid molecules, wherein the first subset of nucleic acid molecules differs from the second subset on nucleic acid molecules by mean size, mean GC content, genomic regions, or a combination thereof.

Certain embodiments may include one or more fragmentation reactions. The fragmentation reactions may comprise fragmenting one or more nucleic acid molecules in a sample or subset of nucleic acid molecules to produce one or more fragmented nucleic acid molecules. The one or more nucleic acid molecules may be fragmented by sonication, needle shear, nebulisation, shearing (e.g., acoustic shearing, mechanical shearing, or point-sink shearing), passage through a French pressure cell, or enzymatic digestion. Enzymatic digestion may occur by nuclease digestion (e.g., micrococcal nuclease digestion, endonucleases, exonucleases, RNase H or DNase I). Fragmentation of the one or more nucleic acid molecules may result in fragment sizes of about 100 base pairs to about 2000 base pairs, about 200 base pairs to about 1500 base pairs, about 200 base pairs to about 1000 base pairs, about 200 base pairs to about 500 base pairs, about 500 base pairs to about 1500 base pairs, and about 500 base pairs to about 1000 base pairs. The one or more fragmentation reactions may result in fragment sizes of about 50 base pairs to about 1000 base pairs. The one or more fragmentation reactions may result in fragment sizes of about 100 base pairs, 150 base pairs, 200 base pairs, 250 base pairs, 300 base pairs, 350 base pairs, 400 base pairs, 450 base pairs, 500 base pairs, 550 base pairs, 600 base pairs, 650 base pairs, 700 base pairs, 750 base pairs, 800 base pairs, 850 base pairs, 900 base pairs, 950 base pairs, 1000 base pairs or more.

Fragmenting the one or more nucleic acid molecules may comprise mechanical shearing of the one or more nucleic acid molecules in the sample for a period of time. The fragmentation reaction may occur for at least about 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more seconds.

Fragmenting the one or more nucleic acid molecules may comprise contacting a nucleic acid sample with one or more beads. Fragmenting the one or more nucleic acid molecules may comprise contacting the nucleic acid sample with a plurality of beads, wherein the ratio of the volume of the plurality of beads to the volume of nucleic acid sample is about 0.10, 0.20, 0.30, 0.40, 0.50, 0.60, 0.70, 0.80, 0.90, 1.00, 1.10, 1.20, 1.30, 1.40, 1.50, 1.60, 1.70, 1.80, 1.90, 2.00 or more. Fragmenting the one or more nucleic acid molecules may comprise contacting the nucleic acid sample with a plurality of beads, wherein the ratio of the volume of the plurality of beads to the volume of nucleic acid is about 2.00, 1.90, 1.80, 1.70, 1.60, 1.50, 1.40, 1.30, 1.20, 1.10, 1.00, 0.90, 0.80, 0.70, 0.60, 0.50, 0.40, 0.30, 0.20, 0.10, 0.05, 0.04, 0.03, 0.02, 0.01 or less.

Certain embodiments may include conducting one or more detection reactions on one or more nucleic acid molecules in a sample. Detection reactions may comprise one or more sequencing reactions. Alternatively, conducting a detection reaction comprises optical sensing, electrical sensing, or a combination thereof. Optical sensing may comprise optical sensing of a photoluminescent photon emission, fluorescence photon emission, pyrophosphate photon emission, chemiluminescence photon emission, or a combination thereof. Electrical sensing may comprise electrical sensing of an ion concentration, ion current modulation, nucleotide electrical field, nucleotide tunneling current, or a combination thereof.

Certain embodiments may include conducting one or more quantification reactions on one or more nucleic acid molecules in a sample. Quantification reactions may comprise sequencing, PCR, qPCR, digital PCR, or a combination thereof.

Certain embodiments may include one or more samples. Certain embodiments may include 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100 or more samples. The sample may be derived from a subject. The two or more samples may be derived from a single subject. The two or more samples may be derived from 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100 or more different subjects. The subject may be a mammal, reptile, amphibian, avian, or fish. The mammal may be a human, ape, orangutan, monkey, chimpanzee, cow, pig, horse, rodent, bird, reptile, dog, cat, or other animal. A reptile may be a lizard, snake, alligator, turtle, crocodile, or tortoise. An amphibian may be a toad, frog, newt, or salamander. Examples of avians include, but are not limited to, ducks, geese, penguins, ostriches, or owls. Examples of fish include, but are not limited to, catfish, eels, sharks, or swordfish. Preferably, the subject is a human. The subject may suffer from a disease or condition (e.g., a cancer).

The two or more samples may be collected over 1, 2, 3, 4, 5, 6, 7, 8, 9, 10, 11, 12, 15, 20, 30, 40, 50, 60, 70, 80, 90, 100, 200, 300, 400, 500, 600, 700, 800, 900, 1000 or time points. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more hour period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more day period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more week period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more month period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more year period.

The sample may be from a body fluid, cell, skin, tissue, organ, or combination thereof. The sample may be a blood, plasma, a blood fraction, saliva, sputum, urine, semen, transvaginal fluid, cerebrospinal fluid, stool, a cell or a tissue biopsy. The sample may be from an adrenal gland, appendix, bladder, brain, ear, esophagus, eye, gall bladder, heart, kidney, large intestine, liver, lung, mouth, muscle, nose, pancreas, parathyroid gland, pineal gland, pituitary gland, skin, small intestine, spleen, stomach, thymus, thyroid gland, trachea, uterus, vermiform appendix, cornea, skin, heart valve, artery, or vein.

The samples may comprise one or more nucleic acid molecules. The nucleic acid molecule may be a DNA molecule, RNA molecule (e.g., mRNA, CRNA or miRNA), or DNA/RNA hybrids. Examples of DNA molecules include, but are not limited to, double-stranded DNA, single-stranded DNA, single-stranded DNA hairpins, cDNA, and genomic DNA. The nucleic acid may be an RNA molecule, such as a double-stranded RNA, single-stranded RNA, ncRNA, RNA hairpin, or mRNA. Examples of ncRNA include, but are not limited to, siRNA, miRNA, snoRNA, piRNA, tiRNA, PASR, TASR, aTASR, TSSa-RNA, snRNA, RE-RNA, uaRNA, x-ncRNA, hY RNA, usRNA, snaR, and vtRNA.

Certain embodiments may include one or more containers. Certain embodiments may include 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more containers. The one or more containers may be different, similar, identical, or a combination thereof. Examples of containers include, but are not limited to, plates, microplates, PCR plates, wells, microwells, tubes, Eppendorf tubes, vials, arrays, microarrays, and chips.

Certain embodiments may include one or more reagents. Certain embodiments may include 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more reagents. The one or more reagents may be different, similar, identical, or a combination thereof. The reagents may improve the efficiency of the one or more assays. Reagents may improve the stability of the nucleic acid molecule or variant or derivative thereof. Reagents may include, but are not limited to, enzymes, proteases, nucleases, molecules, polymerases, reverse transcriptases, ligases, and chemical compounds. Certain embodiments may include conducting an assay comprising one or more antioxidants. Generally, antioxidants are molecules that inhibit oxidation of another molecule. Examples of antioxidants include, but are not limited to, ascorbic acid (e.g., vitamin C), glutathione, lipoic acid, uric acid, carotenes, a-tocopherol (e.g., vitamin E), ubiquinol (e.g., coenzyme Q), and vitamin A.

Certain embodiments may include one or more buffers or solutions. The one or more buffers or solutions may be different, similar, identical, or a combination thereof. The buffers or solutions may improve the efficiency of the one or more assays. Buffers or solutions may improve the stability of the nucleic acid molecule or variant or derivative thereof. Buffers or solutions may include, but are not limited to, wash buffers, elution buffers, and hybridization buffers.

Certain embodiments may include one or more beads, a plurality of beads, or one or more bead sets. Certain embodiments may include 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more. 500 or more, 600 or more, 700 or more, 800 or more, 900 or more, or 1000 or more one or more beads or bead sets. The one or more beads or bead sets may be different, similar, identical, or a combination thereof. The beads may be magnetic, antibody coated, protein A crosslinked, protein G crosslinked, streptavidin coated, oligonucleotide conjugated, silica coated, or a combination thereof. Examples of beads include, but are not limited to, AMPure beads, AMPure XP beads, streptavidin beads, agarose beads, magnetic beads, Dynabeads®, MACS® microbeads, antibody conjugated beads (e.g, anti-immunoglobulin microbeads), protein A conjugated beads, protein G conjugated beads, protein A/G conjugated beads, protein L conjugated beads, oligo-dT conjugated beads, silica beads, silica-like beads, anti-biotin microbeads, anti-fluorochrome microbeads, and BcMag™ Carboxy-Terminated Magnetic Beads. In some aspects of the disclosure, the one or more beads comprise one or more AMPure beads. Alternatively. or additionally, the one or more beads comprise AMPure XP beads.

Certain embodiments may include one or more primers, a plurality of primers, or one or more primer sets. The primers may further comprise one or more linkers. The primers may further comprise or more labels. The primers may be used in one or more assays. For example, the primers are used in one or more sequencing reactions, amplification reactions, or a combination thereof. Certain embodiments may include 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more one or more primers or primer sets. The primers may comprise about 100 nucleotides. The primers may comprise between about 10 to about 500 nucleotides, between about 20 to about 450 nucleotides, between about 30 to about 400 nucleotides, between about 40 to about 350 nucleotides, between about 50 to about 300 nucleotides, between about 60 to about 250 nucleotides, between about 70 to about 200 nucleotides, or between about 80 to about 150 nucleotides. In some aspects of the disclosure, the primers comprise between about 80 nucleotides to about 100 nucleotides. The one or more primers or primer sets may be different, similar, identical, or a combination thereof.

The primers may hybridize to at least a portion of the one or more nucleic acid molecules or variant or derivative thereof in the sample or subset of nucleic acid molecules. The primers may hybridize to one or more genomic regions. The primers may hybridize to different, similar, and/or identical genomic regions. The one or more primers may be at least about 50%, 55%, 60%, 65%, 70%, 75%, 80%, 85%, 90%, 95%, 97%, 99% or more complementary to the one or more nucleic acid molecules or variant or derivative thereof.

The primers may comprise one or more nucleotides. The primers may comprise 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more nucleotides. The primers may comprise about 100 nucleotides. The primers may comprise between about 10 to about 500 nucleotides, between about 20 to about 450 nucleotides, between about 30 to about 400 nucleotides, between about 40 to about 350 nucleotides, between about 50 to about 300 nucleotides, between about 60 to about 250 nucleotides, between about 70 to about 200 nucleotides, or between about 80 to about 150 nucleotides. In some aspects of the disclosure, the primers comprise between about 80 nucleotides to about 100 nucleotides.

The plurality of primers or the primer sets may comprise two or more primers with identical, similar, and/or different sequences, linkers, and/or labels. For example, two or more primers comprise identical sequences. In another example, two or more primers comprise similar sequences. In yet another example, two or more primers comprise different sequences. The two or more primers may further comprise one or more linkers. The two or more primers may further comprise different linkers. The two or more primers may further comprise similar linkers. The two or more primers may further comprise identical linkers. The two or more primers may further comprise one or more labels. The two or more primers may further comprise different labels. The two or more primers may further comprise similar labels. The two or more primers may further comprise identical labels.

The capture probes, primers, labels, and/or beads may comprise one or more nucleotides. The one or more nucleotides may comprise RNA, DNA, a mix of DNA and RNA residues or their modified analogs such as 2′-OMe, or 2′-fluoro (2′-F), locked nucleic acids (LNA), or abasic sites.

Certain embodiments may include one or more labels. Certain embodiments may include 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more one or more labels. The one or more labels may be different, similar, identical, or a combination thereof.

Examples of labels include, but are not limited to, chemical, biochemical, biological, colorimetric, enzymatic, fluorescent, and luminescent labels, which are well known in the art. The label comprise a dye, a photocrosslinker, a cytotoxic compound, a drug, an affinity label, a photoaffinity label, a reactive compound, an antibody or antibody fragment, a biomaterial, a nanoparticle, a spin label, a fluorophore, a metal-containing moiety, a radioactive moiety, a novel functional group, a group that covalently or noncovalently interacts with other molecules, a photocaged moiety, an actinic radiation excitable moiety, a ligand, a photoisomerizable moiety, biotin, a biotin analogue, a moiety incorporating a heavy atom, a chemically cleavable group, a photocleavable group, a redox-active agent, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, or a combination thereof.

The label may be a chemical label. Examples of chemical labels can include, but are not limited to, biotin and radioisotopes (e.g., iodine, carbon, phosphate, or hydrogen).

The methods, kits, and compositions disclosed herein may comprise a biological label. The biological labels may comprise metabolic labels, including, but not limited to, bioorthogonal azide-modified amino acids, sugars, and other compounds.

The methods, kits, and compositions disclosed herein may comprise an enzymatic label. Enzymatic labels can include but are not limited to: horseradish peroxidase (HRP), alkaline phosphatase (AP), glucose oxidase, and 0-galactosidase. The enzymatic label may be luciferase.

The methods, kits, and compositions disclosed herein may comprise a fluorescent label. The fluorescent label may be an organic dye (e.g., FITC), biological fluorophore (e.g., green fluorescent protein), or quantum dot. A non-limiting list of fluorescent labels includes fluorescein isothiocyante (FITC), DyLight Fluors, fluorescein, rhodamine (tetramethyl rhodamine isothiocyanate, TRITC), coumarin, Lucifer Yellow, and BODIPY. The label may be a fluorophore. Exemplary fluorophores include, but are not limited to, indocarbocyanine (C3), indodicarbocyanine (C5), Cy3, Cy3.5, Cy5, Cy5.5, Cy7, Texas Red, Pacific Blue, Oregon Green 488, Alexa Fluor® 355, Alexa Fluor 488, Alexa Fluor 532, Alexa Fluor 546, Alexa Fluor 555, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROXTM), LIZTM, VICTM, NEDTM, PETTM, SYBR, PicoGreen, RiboGreen, and the like. The fluorescent label may be a green fluorescent protein (GFP), red fluorescent protein (RFP), yellow fluorescent protein, phycobiliproteins (e.g., allophycocyanin, phycocyanin, phycoerythrin, or phycoerythrocyanin).

Certain embodiments may include one or more linkers. Certain embodiments may include 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more one or more linkers. The one or more linkers may be different, similar, identical, or a combination thereof.

Suitable linkers comprise any chemical or biological compound capable of attaching to a label, primer, and/or capture probe disclosed herein. If the linker attaches to both the label and the primer or capture probe, then a suitable linker would be capable of sufficiently separating the label and the primer or capture probe. Suitable linkers would not significantly interfere with the ability of the primer and/or capture probe to hybridize to a nucleic acid molecule, portion thereof, or variant or derivative thereof. Suitable linkers would not significantly interfere with the ability of the label to be detected. The linker may be rigid. The linker may be flexible. The linker may be semi rigid. The linker may be proteolytically stable (e.g., resistant to proteolytic cleavage). The linker may be proteolytically unstable (e.g., sensitive to proteolytic cleavage). The linker may be helical. The linker may be non-helical. The linker may be coiled. The linker may be 3-stranded. The linker may comprise a turn conformation. The linker may be a single chain. The linker may be a long chain. The linker may be a short chain. The linker may comprise at least about 5 residues, at least about 10 residues, at least about 15 residues, at least about 20 residues, at least about 25 residues, at least about 30 residues, or at least about 40 residues or more.

Examples of linkers include, but are not limited to, hydrazone, disulfide, thioether, and peptide linkers. The linker may be a peptide linker. The peptide linker may comprise a proline residue. The peptide linker may comprise an arginine, phenylalanine, threonine, glutamine, glutamate, or any combination thereof. The linker may be a heterobifunctional crosslinker.

Certain embodiments may include conducting 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 20 or more, 25 or more, 30 or more, 35 or more, 40 or more, 45 or more, or 50 or more assays on a sample comprising one or more nucleic acid molecules. the two or more assays may be different, similar, identical, or a combination thereof. For example, certain embodiments comprise conducting two or more sequencing reactions. In another example, certain embodiments comprise conducting two or more assays, wherein at least one of the two or more assays comprises a sequencing reaction. In yet another example, certain embodiments comprise conducting two or more assays, wherein at least two of the two or more assays comprise a sequencing reaction and a hybridization reaction. The two or more assays may be performed sequentially, simultaneously, or a combination thereof. For example, the two or more sequencing reactions may be performed simultaneously. In another example, certain embodiments comprise conducting a hybridization reaction, followed by a sequencing reaction. In yet another example, certain embodiments comprise conducting two or more hybridization reactions simultaneously, followed by conducting two or more sequencing reactions simultaneously. The two or more assays may be performed by one or more devices. For example, two or more amplification reactions may be performed by a PCR machine. In another example, two or more sequencing reactions may be performed by two or more sequencers.

### XII. Assays and Amplification Techniques

Certain embodiments may include conducting one or more assays on a sample comprising one or more nucleic acid molecules. Producing two or more subsets of nucleic acid molecules may comprise conducting one or more assays. The assays may be conducted on a subset of nucleic acid molecules from the sample. The assays may be conducted on one or more nucleic acids molecules from the sample. The assays may be conducted on at least a portion of a subset of nucleic acid molecules. The assays may comprise one or more techniques, reagents, capture probes, primers, labels, and/or components for the detection, quantification, and/or analysis of one or more nucleic acid molecules.

Certain embodiments may include one or more sequencers. The one or more sequencers may comprise one or more HiSeq, MiSeq, HiScan, NovaSeq, PacBio RS, RSII, Sequel, Sequel II, Element Biosciences Aviti, Genapsys Sequencer, Genome Analyzer IIx, SOLID Sequencer, Ion Torrent PGM, 454 GS Junior, Pac Bio RS, Ultima Genomics UG 100, PacBio Revio, PacBio Onso, another existing or future sequencer, or a combination thereof. The one or more sequencers may comprise one or more sequencing platforms. The one or more sequencing platforms may comprise GS FLX by 454 Life Technologies/Roche, Genome Analyzer by Solexa/Illumina, SOLID by Applied Biosystems, CGA Platform by Complete Genomics, PacBio RS by Pacific Biosciences, or a combination thereof.

Certain embodiments may include one or more thermocyclers. The one or more

thermocyclers may be used to amplify one or more nucleic acid molecules. Certain embodiments may include one or more real-time PCR instruments. The one or more real-time PCR instruments may comprise a thermal cycler and a fluorimeter. The one or more thermocyclers may be used to amplify and detect one or more nucleic acid molecules.

Certain embodiments may include one or more magnetic separators. The one or more magnetic separators may be used for separation of paramagnetic and ferromagnetic particles from a suspension. The one or more magnetic separators may comprise one or more LifeStep™ biomagnetic separators, SPHERO™ FlexiMag separator, SPHERO™ MicroMag separator, SPHERO™ HandiMag separator, SPHERO™ MiniTube Mag separator, SPHERO™ UltraMag separator, DynaMag™ magnet, DynaMag™-2 Magnet, or a combination thereof.

Certain embodiments may include one or more bioanalyzers. Generally, a bioanalyzer is a chip-based capillary electrophoresis machine that can analyze RNA, DNA, and proteins. The one or more bioanalyzers may comprise Agilent's 2100 Bioanalyzer, Tapestation 2200, and/or Tapestation 4200.

Certain embodiments may include one or more processors. The one or more processors may analyze, compile, store, sort, combine, assess or otherwise process one or more data and/or results from one or more assays, one or more data and/or results based on or derived from one or more assays, one or more outputs from one or more assays, one or more outputs based on or derived from one or more assays, one or more outputs from one or data and/or results, one or more outputs based on or derived from one or more data and/or results, or a combination thereof. The one or more processors may transmit the one or more data, results, or outputs from one or more assays, one or more data, results, or outputs based on or derived from one or more assays, one or more outputs from one or more data or results, one or more outputs based on or derived from one or more data or results, or a combination thereof. The one or more processors may receive and/or store requests from a user. The one or more processors may produce or generate one or more data, results, outputs. The one or more processors may produce or generate one or more biomedical reports. The one or more processors may transmit one or more biomedical reports. The one or more processors may analyze, compile, store, sort, combine, assess or otherwise process information from one or more databases, one or more data or results, one or more outputs, or a combination thereof. The one or more processors may analyze, compile, store, sort, combine, assess or otherwise process information from 1, 2, 3, 4, 5, 6, 7, 8, 9, 10, 11, 12, 13, 14, 15, 16, 17, 18, 19, 20, 30 or more databases. The one or more processors may transmit one or more requests, data, results, outputs and/or information to one or more users, processors, computers, computer systems, memory locations, devices, databases, or a combination thereof. The one or more processors may receive one or more requests, data, results, outputs and/or information from one or more users, processors, computers, computer systems, memory locations, devices, databases or a combination thereof. The one or more processors may retrieve one or more requests, data, results, outputs and/or information from one or more users, processors, computers, computer systems, memory locations, devices, databases or a combination thereof.

Certain embodiments may include one or more memory locations. The one or more memory locations may store information, data, results, outputs, requests, or a combination thereof. The one or more memory locations may receive information, data, results, outputs, requests, or a combination thereof from one or more users, processors, computers, computer systems, devices, or a combination thereof.

Methods described herein can be implemented with the aid of one or more computers and/or computer systems. A computer or computer system may comprise electronic storage locations (e.g., databases or memory) with machine-executable code for implementing the methods provided herein, and one or more processors for executing the machine-executable code.

The code can be pre-compiled and configured for use with a machine having a processer adapted to execute the code or can be compiled during runtime. The code can be supplied in a programming language that can be selected to enable the code to execute in a pre-compiled or as-compiled fashion.

The one or more computers and/or computer systems may analyze, compile, store, sort, combine, assess or otherwise process one or more data and/or results from one or more assays, one or more data and/or results based on or derived from one or more assays, one or more outputs from one or more assays, one or more outputs based on or derived from one or more assays, one or more outputs from one or more data and/or results, one or more outputs based on or derived from one or more data and/or results, or a combination thereof. The one or more computers and/or computer systems may transmit the one or more data, results, or outputs from one or more assays, one or more data, results, or outputs based on or derived from one or more assays, one or more outputs from one or more data or results, one or more outputs based on or derived from one or more data or results, or a combination thereof. The one or more computers and/or computer systems may receive and/or store requests from a user. The one or more computers and/or computer systems may produce or generate one or more data, results, outputs. The one or more computers and/or computer systems may produce or generate one or more biomedical reports. The one or more computers and/or computer systems may transmit one or more biomedical reports. The one or more computers and/or computer systems may analyze, compile, store, sort, combine, assess or otherwise process information from one or more databases, one or more data or results, one or more outputs, or a combination thereof. The one or more computers and/or computer systems may analyze, compile, store, sort, combine, assess or otherwise process information from 1, 2, 3, 4, 5, 6, 7, 8, 9, 10, 11, 12, 13, 14, 15, 16, 17, 18, 19, 20, 30 or more databases. The one or more computers and/or computer systems may transmit one or more requests, data, results, outputs, and/or information to one or more users, processors, computers, computer systems, memory locations, devices, or a combination thereof. The one or more computers and/or computer systems may receive one or more requests, data, results, outputs, and/or information from one or more users, processors, computers, computer systems, memory locations, devices, or a combination thereof. The one or more computers and/or computer systems may retrieve one or more requests, data, results, outputs and/or information from one or more users, processors, computers, computer systems, memory locations, devices, databases or a combination thereof.

### XIII. Databases

Certain embodiments may include one or more databases. Certain embodiments may include at least about 1, 2, 3, 4, 5, 6, 7, 8, 9, 10, 11, 12, 13, 14, 15, 16, 17, 18, 19, 20, 30 or more databases. The databases may comprise genomic, proteomic, pharmacogenomic, biomedical, or scientific databases. The databases may be publicly available databases. Alternatively, or additionally, the databases may comprise proprietary databases. The databases may be commercially available databases. The databases include, but are not limited to, The Cancer Genomic Atlas, Cosmic, GnomAD, Dbsnp, Mills Indels, MendelDB, PharmGKB, Varimed, Regulome, curated BreakSeq junctions, Online Mendelian Inheritance in Man (OMIM), Human Genome Mutation Database (HGMD), NCBI db SNP, NCBI RefSeq, GENCODE, GO (gene ontology), and Kyoto Encyclopedia of Genes and Genomes (KEGG). The databases may comprise one or more of: (i) population-level data, (ii) subject-specific data, (iii) organ system-specific data, (iv) organ-specific data, (v) tissue-specific data, (vi) cell-type-specific data, (vii) disease-specific data, (viii) cancer-specific data, (ix) polymorphism data, (x) methylation data (e.g., hypomethylation data, hypermethylation data, data regarding the normal methylation status of a particular genomic region or locus, etc.), and the like, as well as any combination thereof. In some instances, the databases may comprise sequencing data. For example, the one or more databases may comprise one or more of: (i) population-level sequencing data, (ii) subject-specific sequencing data, (iii) organ system-specific sequencing data, (iv) organ-specific sequencing data, (v) tissue-specific sequencing data, (vi) cell-type-specific sequencing data, (vii) disease-specific sequencing data, (viii) cancer-specific sequencing data, (xi) data on polymorphisms derived from sequencing, (x) data on methylation status or state derived from sequencing, and the like, as well as any combination thereof.

Certain embodiments may include analyzing one or more databases. Certain embodiments may include analyzing at least about 1, 2, 3, 4, 5, 6, 7, 8, 9, 10, 11, 12, 13, 14, 15, 16, 17, 18, 19, 20, 30 or more databases. Analyzing the one or more databases may comprise one or more algorithms, computers, processors, memory locations, devices, or a combination thereof.

Certain embodiments may include identifying one or more nucleic acid regions based on data and/or information from one or more databases. Certain embodiments may include identifying one or more sets of nucleic acid regions based on data and/or information from one or more databases. Certain embodiments may include identifying one or more nucleic acid regions and/or sets of nucleic acid regions based on data and/or information from at least about 2 or more databases. Certain embodiments may include identifying one or more nucleic acid regions and/or sets of nucleic acid regions based on data and/or information from at least about 3 or more databases. Certain embodiments may include identifying one or more nucleic acid regions and/or sets of nucleic acid regions based on data and/or information from at least about 4, 5, 6, 7, 8, 9, 10, 11, 12, 13, 14, 15, 16, 17, 18, 19, 20, 30 or more databases.

Certain embodiments may include analyzing one or more results based on data and/or information from one or more databases. Certain embodiments may include analyzing one or more sets of results based on data and/or information from one or more databases. Certain embodiments may include analyzing one or more combined results based on data and/or information from one or more databases. Certain embodiments may include analyzing one or more results, sets of results, and/or combined results based on data and/or information from at least about 2 or more databases. Certain embodiments may include analyzing one or more results, sets of results, and/or combined results based on data and/or information from at least about 3 or more databases. Certain embodiments may include analyzing one or more results, sets of results, and/or combined results based on data and/or information from at least about 4, 5, 6, 7, 8, 9, 10, 11, 12, 13, 14, 15, 16, 17, 18, 19, 20, 30 or more databases.

Certain embodiments may include comparing one or more results based on data and/or information from one or more databases. Certain embodiments may include comparing one or more sets of results based on data and/or information from one or more databases. Certain embodiments may include comparing one or more combined results based on data and/or information from one or more databases. Certain embodiments may include comparing one or more results, sets of results, and/or combined results based on data and/or information from at least about 2 or more databases. Certain embodiments may include comparing one or more results, sets of results, and/or combined results based on data and/or information from at least about 3 or more databases. Certain embodiments may include comparing one or more results, sets of results, and/or combined results based on data and/or information from at least about 4, 5, 6, 7, 8, 9, 10, 11, 12, 13, 14, 15, 16, 17, 18, 19, 20, 30 or more databases.

Certain embodiments may include biomedical databases, genomic databases, biomedical reports, disease reports, case-control analysis, and rare variant discovery analysis based on data and/or information from one or more databases, one or more assays, one or more data or results, one or more outputs based on or derived from one or more assays, one or more outputs based on or derived from one or more data or results, or a combination thereof.

### XIV. Datasets and Analysis

Certain embodiments may include one or more data, one or more data sets, one or more combined data, one or more combined data sets, one or more results, one or more sets of results, one or more combined results, or a combination thereof. The data and/or results may be based on or derived from one or more assays, one or more databases, or a combination thereof. Certain embodiments may include analysis of the one or more data, one or more data sets, one or more combined data, one or more combined data sets, one or more results, one or more sets of results, one or more combined results, or a combination thereof. Certain embodiments may include processing of the one or more data, one or more data sets, one or more combined data, one or more combined data sets, one or more results, one or more sets of results, one or more combined results, or a combination thereof.

Certain embodiments may include at least one analysis and at least one processing of the one or more data, one or more data sets, one or more combined data, one or more combined data sets, one or more results, one or more sets of results, one or more combined results, or a combination thereof. Certain embodiments may include one or more analyses and one or more processing of the one or more data, one or more data sets, one or more combined data, one or more combined data sets, one or more results, one or more sets of results, one or more combined results, or a combination thereof. Certain embodiments may include at least 1, 2, 3, 4, 5, 6, 7, 8, 9, 10, 15, 20, 30, 40, 50, 60, 70, 80, 90, 100, 200, 300, 400, 500, 600, 700, 800, 900, 1000 or more distinct analyses of the one or more data, one or more data sets, one or more combined data, one or more combined data sets, one or more results, one or more sets of results, one or more combined results, or a combination thereof. Certain embodiments may include at least 1, 2, 3, 4, 5, 6, 7, 8, 9, 10, 15, 20, 30, 40, 50, 60, 70, 80, 90, 100, 200, 300, 400, 500, 600, 700, 800, 900, 1000 or more distinct processing of the one or more data, one or more data sets, one or more combined data, one or more combined data sets, one or more results, one or more sets of results, one or more combined results, or a combination thereof. The one or more analyses and/or one or more processing may occur simultaneously, sequentially, or a combination thereof.

The one or more analyses and/or one or more processing may occur over 1, 2, 3, 4, 5, 6, 7, 8, 9, 10, 11, 12, 15, 20, 30, 40, 50, 60, 70, 80, 90, 100, 200, 300, 400, 500, 600, 700, 800, 900, 1000 or time points. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more hour period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more day period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more week period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more month period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more year period.

Certain embodiments may include one or more data. The one or more data may comprise one or more raw data based on or derived from one or more assays. The one or more data may comprise one or more raw data based on or derived from one or more databases. The one or more data may comprise at least partially analyzed data based on or derived from one or more raw data. The one or more data may comprise at least partially processed data based on or derived from one or more raw data. The one or more data may comprise fully analyzed data based on or derived from one or more raw data. The one or more data may comprise fully processed data based on or derived from one or more raw data. The data may comprise sequencing read data or expression data. The data may comprise biomedical, scientific, pharmacological, and/or genetic information.

Certain embodiments may include one or more combined data. The one or more combined data may comprise two or more data. The one or more combined data may comprise two or more data sets. The one or more combined data may comprise one or more raw data based on or derived from one or more assays. The one or more combined data may comprise one or more raw data based on or derived from one or more databases. The one or more combined data may comprise at least partially analyzed data based on or derived from one or more raw data. The one or more combined data may comprise at least partially processed data based on or derived from one or more raw data. The one or more combined data may comprise fully analyzed data based on or derived from one or more raw data. The one or more combined data may comprise fully processed data based on or derived from one or more raw data. One or more combined data may comprise sequencing read data or expression data. One or more combined data may comprise biomedical, scientific, pharmacological, and/or genetic information.

Certain embodiments may include one or more data sets. The one or more data sets may comprise one or more data. The one or more data sets may comprise one or more combined data. The one or more data sets may comprise one or more raw data based on or derived from one or more assays. The one or more data sets may comprise one or more raw data based on or derived from one or more databases. The one or more data sets may comprise at least partially analyzed data based on or derived from one or more raw data. The one or more data sets may comprise at least partially processed data based on or derived from one or more raw data. The one or more data sets may comprise fully analyzed data based on or derived from one or more raw data. The one or more data sets may comprise fully processed data based on or derived from one or more raw data. The data sets may comprise sequencing read data or expression data. The data sets may comprise biomedical, scientific, pharmacological, and/or genetic information.

Certain embodiments may include one or more combined data sets. The one or more combined data sets may comprise two or more data. The one or more combined data sets may comprise two or more combined data. The one or more combined data sets may comprise two or more data sets. The one or more combined data sets may comprise one or more raw data based on or derived from one or more assays. The one or more combined data sets may comprise one or more raw data based on or derived from one or more databases. The one or more combined data sets may comprise at least partially analyzed data based on or derived from one or more raw data. The one or more combined data sets may comprise at least partially processed data based on or derived from one or more raw data. The one or more combined data sets may comprise fully analyzed data based on or derived from one or more raw data. The one or more combined data sets may comprise fully processed data based on or derived from one or more raw data. Certain embodiments may further comprise further processing and/or analysis of the combined data sets. One or more combined data sets may comprise sequencing read data or expression data. One or more combined data sets may comprise biomedical, scientific, pharmacological, and/or genetic information.

Certain embodiments may include one or more results. The one or more results may comprise one or more data, data sets, combined data, and/or combined data sets. The one or more results may be based on or derived from one or more data, data sets, combined data, and/or combined data sets. The one or more results may be produced from one or more assays. The one or more results may be based on or derived from one or more assays. The one or more results may be based on or derived from one or more databases. The one or more results may comprise at least partially analyzed results based on or derived from one or more data, data sets, combined data, and/or combined data sets. The one or more results may comprise at least partially processed results based on or derived from one or more data. data sets, combined data, and/or combined data sets. The one or more results may comprise fully analyzed results based on or derived from one or more data, data sets, combined data, and/or combined data sets. The one or more results may comprise fully processed results based on or derived from one or more data, data sets, combined data, and/or combined data sets. The results may comprise sequencing read data or expression data. The results may comprise biomedical, scientific, pharmacological, and/or genetic information.

Certain embodiments may include one or more sets of results. The one or more sets of results may comprise one or more data, data sets, combined data, and/or combined data sets. The one or more sets of results may be based on or derived from one or more data, data sets, combined data, and/or combined data sets. The one or more sets of results may be produced from one or more assays. The one or more sets of results may be based on or derived from one or more assays. The one or more sets of results may be based on or derived from one or more databases. The one or more sets of results may comprise at least partially analyzed sets of results based on or derived from one or more data, data sets, combined data, and/or combined data sets. The one or more sets of results may comprise at least partially processed sets of results based on or derived from one or more data, data sets, combined data, and/or combined data sets. The one or more sets of results may comprise fully analyzed sets of results based on or derived from one or more data, data sets, combined data, and/or combined data sets. The one or more sets of results may comprise fully processed sets of results based on or derived from one or more data, data sets, combined data, and/or combined data sets. The sets of results may comprise sequencing read data or expression data. The sets of results may comprise biomedical, scientific, pharmacological, and/or genetic information.

Certain embodiments may include one or more combined results. The combined results may comprise one or more results, sets of results, and/or combined sets of results. The combined results may be based on or derived from one or more results, sets of results, and/or combined sets of results. The one or more combined results may comprise one or more data, data sets, combined data, and/or combined data sets. The one or more combined results may be based on or derived from one or more data, data sets, combined data, and/or combined data sets. The one or more combined results may be produced from one or more assays. The one or more combined results may be based on or derived from one or more assays. The one or more combined results may be based on or derived from one or more databases. The one or more combined results may comprise at least partially analyzed combined results based on or derived from one or more data, data sets, combined data, and/or combined data sets. The one or more combined results may comprise at least partially processed combined results based on or derived from one or more data, data sets, combined data, and/or combined data sets. The one or more combined results may comprise fully analyzed combined results based on or derived from one or more data, data sets, combined data, and/or combined data sets. The one or more combined results may comprise fully processed combined results based on or derived from one or more data, data sets, combined data, and/or combined data sets. The combined results may comprise sequencing read data or expression data. The combined results may comprise biomedical, scientific, pharmacological, and/or genetic information.

Certain embodiments may include one or more combined sets of results. The combined sets of results may comprise one or more results, sets of results, and/or combined results. The combined sets of results may be based on or derived from one or more results, sets of results, and/or combined results. The one or more combined sets of results may comprise one or more data, data sets, combined data, and/or combined data sets. The one or more combined sets of results may be based on or derived from one or more data, data sets, combined data, and/or combined data sets. The one or more combined sets of results may be produced from one or more assays. The one or more combined sets of results may be based on or derived from one or more assays. The one or more combined sets of results may be based on or derived from one or more databases. The one or more combined sets of results may comprise at least partially analyzed combined sets of results based on or derived from one or more data, data sets, combined data, and/or combined data sets. The one or more combined sets of results may comprise at least partially processed combined sets of results based on or derived from one or more data, data sets, combined data, and/or combined data sets. The one or more combined sets of results may comprise fully analyzed combined sets of results based on or derived from one or more data, data sets, combined data, and/or combined data sets. The one or more combined sets of results may comprise fully processed combined sets of results based on or derived from one or more data, data sets, combined data, and/or combined data sets. The combined sets of results may comprise sequencing read data or expression data. The combined sets of results may comprise biomedical, scientific, pharmacological, and/or genetic information.

Certain embodiments may include one or more outputs, sets of outputs, combined outputs, and/or combined sets of outputs. The methods, libraries, kits and systems herein may comprise producing one or more outputs, sets of outputs, combined outputs, and/or combined sets of outputs. The sets of outputs may comprise one or more outputs, one or more combined outputs, or a combination thereof. The combined outputs may comprise one or more outputs, one or more sets of outputs, one or more combined sets of outputs, or a combination thereof. The combined sets of outputs may comprise one or more outputs, one or more sets of outputs, one or more combined outputs, or a combination thereof. The one or more outputs, sets of outputs, combined outputs, and/or combined sets of outputs may be based on or derived from one or more data, one or more data sets, one or more combined data, one or more combined data sets, one or more results, one or more sets of results, one or more combined results, or a combination thereof. The one or more outputs, sets of outputs, combined outputs, and/or combined sets of outputs may be based on or derived from one or more databases. The one or more outputs, sets of outputs, combined outputs, and/or combined sets of outputs may comprise one or more biomedical reports, biomedical outputs, rare variant outputs, pharmacogenetic outputs, population study outputs, case-control outputs, biomedical databases, genomic databases, disease databases, net content.

Certain embodiments may include one or more biomedical outputs, one or more sets of biomedical outputs, one or more combined biomedical outputs, one or more combined sets of biomedical outputs. The methods, libraries, kits and systems herein may comprise producing one or more biomedical outputs, one or more sets of biomedical outputs, one or more combined biomedical outputs, one or more combined sets of biomedical outputs. The sets of biomedical outputs may comprise one or more biomedical outputs, one or more combined biomedical outputs, or a combination thereof. The combined biomedical outputs may comprise one or more biomedical outputs, one or more sets of biomedical outputs, one or more combined sets of biomedical outputs, or a combination thereof. The combined sets of biomedical outputs may comprise one or more biomedical outputs, one or more sets of biomedical outputs, one or more combined biomedical outputs, or a combination thereof. The one or more biomedical outputs, one or more sets of biomedical outputs, one or more combined biomedical outputs, one or more combined sets of biomedical outputs may be based on or derived from one or more data, one or more data sets, one or more combined data, one or more combined data sets, one or more results, one or more sets of results, one or more combined results, one or more outputs, one or more sets of outputs, one or more combined outputs, one or more sets of combined outputs, or a combination thereof. The one or more biomedical outputs may comprise biomedical information of a subject. The biomedical information of the subject may predict, diagnose, and/or prognose one or more biomedical features. The one or more biomedical features may comprise the status of a disease or condition, genetic risk of a disease or condition, reproductive risk, genetic risk to a fetus, risk of an adverse drug reaction, efficacy of a drug therapy, prediction of optimal drug dosage, transplant tolerance, or a combination thereof.

Certain embodiments may include one or more biomedical reports. The methods, libraries, kits and systems herein may comprise producing one or more biomedical reports. The one or more biomedical reports may be based on or derived from one or more data, one or more data sets, one or more combined data, one or more combined data sets, one or more results, one or more sets of results, one or more combined results, one or more outputs, one or more sets of outputs, one or more combined outputs, one or more sets of combined outputs, one or more biomedical outputs, one or more sets of biomedical outputs, combined biomedical outputs, one or more sets of biomedical outputs, or a combination thereof. The biomedical report may predict, diagnose, and/or prognose one or more biomedical features. The one or more biomedical features may comprise the status of a disease or condition, genetic risk of a disease or condition, reproductive risk, genetic risk to a fetus, risk of an adverse drug reaction, efficacy of a drug therapy, prediction of optimal drug dosage, transplant tolerance, or a combination thereof.

Certain embodiments may also comprise the transmission of one or more data, information, results, outputs, reports or a combination thereof. For example, data/information based on or derived from the one or more assays are transmitted to another device and/or instrument. In another example, the data, results, outputs, biomedical outputs, biomedical reports, or a combination thereof are transmitted to another device and/or instrument. The information obtained from an algorithm may also be transmitted to another device and/or instrument. Information based on the analysis of one or more databases may be transmitted to another device and/or instrument. Transmission of the data/information may comprise the transfer of data/information from a first source to a second source. The first and second sources may be in the same approximate location (e.g., within the same room, building, block, or campus). Alternatively, first and second sources may be in multiple locations (e.g., multiple cities, states, countries, continents, etc.). The data, results, outputs, biomedical outputs, biomedical reports can be transmitted to a patient and/or a healthcare provider.

Transmission may be based on the analysis of one or more data, results, information, databases, outputs, reports, or a combination thereof. For example, transmission of a second report is based on the analysis of a first report. Alternatively, transmission of a report is based on the analysis of one or more data or results. Transmission may be based on receiving one or more requests. For example, transmission of a report may be based on receiving a request from a user (e.g., a patient, healthcare provider, or individual).

Transmission of the data/information may comprise digital transmission or analog transmission. Digital transmission may comprise the physical transfer of data (a digital bit stream) over a point-to-point or point-to-multipoint communication channel. Examples of such channels are copper wires, optical fibers, wireless communication channels, and storage media. The data may be represented as an electromagnetic signal, such as an electrical voltage, radio wave, microwave, or infrared signal.

Analog transmission may comprise the transfer of a continuously varying analog signal. The messages can either be represented by a sequence of pulses by means of a line code (baseband transmission), or by a limited set of continuously varying wave forms (passband transmission), using a digital modulation method. The passband modulation and corresponding demodulation (also known as detection) can be carried out by modem equipment. According to the most common definition of digital signal, both baseband and passband signals representing bit-streams are considered as digital transmission, while an alternative definition only considers the baseband signal as digital, and passband transmission of digital data as a form of digital-to-analog conversion.

Certain embodiments may include one or more sample identifiers. The sample identifiers may comprise labels, barcodes, and other indicators which can be linked to one or more samples and/or subsets of nucleic acid molecules. Certain embodiments may include one or more processors, one or more memory locations, one or more computers, one or more monitors, one or more computer software, one or more algorithms for linking data, results, outputs, biomedical outputs, and/or biomedical reports to a sample.

Certain embodiments may include a processor for correlating the expression levels of one or more nucleic acid molecules with a prognosis of disease outcome. Certain embodiments may include one or more of a variety of correlative techniques, including lookup tables, algorithms, multivariate models, and linear or nonlinear combinations of expression models or algorithms. The expression levels may be converted to one or more likelihood scores, reflecting a likelihood that the patient providing the sample may exhibit a particular disease outcome. The models and/or algorithms can be provided in machine readable format and can optionally further designate a treatment modality for a patient or class of patients.

In some instances, the methods and systems as described herein are used to generate an output comprising detection and/or quantitation of genomic DNA regions such as a region containing a DNA polymorphism (e.g., a germline variant or a somatic variant). In some instances, the detection of the one or more genomic regions is based on one or more algorithms, depending on the source of data inputs or databases that are described elsewhere in the instant specification. Each of the one or more algorithms can be used to receive, combine and generate data comprising detection of genomic regions (i.e., polymorphisms). In some embodiments, the instant method and system can comprise detection of the genomic regions that is based on one or more, two or more, three or more, four or more, five or more, six or more, seven or more, eight or more, nine or more or ten or more algorithms. The algorithms can be machine-learning algorithms, computer-implemented algorithms, machine-executed algorithms, automatic algorithms and the like.

The resulting data for each nucleic acid sample can be analyzed using feature selection techniques including filter techniques which assess the relevance of features by examining the intrinsic properties of the data, wrapper methods which embed the model hypothesis within a feature subset search, and embedded techniques in which the search for an optimal set of features is built into an algorithm or model.

In some instances, the detection of the one or more genomic regions is based on one or more statistical models. Statistical models or filtering techniques useful in the methods of the present invention include (1) parametric methods such as the use of two sample t-tests, ANOVA analyses, Bayesian frameworks, and Gamma distribution models, (2) model free methods such as the use of Wilcoxon rank sum tests, between-within class sum of squares tests, rank products methods, random permutation methods, or TNOM which involves setting a threshold point for fold-change differences in expression between two datasets and then detecting the threshold point in each gene that minimizes the number of misclassifications, and (3) multivariate methods such as bivariate methods, correlation based feature selection methods (CFS), minimum redundancy maximum relevance methods (MRMR), Markov blanket filter methods, Markov models, Hidden Markov Models (HMM), and uncorrelated shrunken centroid methods. In some instances, the Hidden Markov Model (HMM) is given an internal state, wherein the internal state is set according to an overall copy number of a chromosome in the first or second nucleic acid sample. In an instance, for a diploid chromosome, the HMM's internal states can be homozygous deletion (locally zero copies), heterozygous deletion (locally one copy), normal (locally two copies), duplication (more than two copies), and reference gap (present as a state to distinguish gaps from homozygous deletions). In another instance, for a haploid chromosome (e.g., X or Yin a male), the HMIM's internal states can be homozygous deletion (locally zero copies), normal (locally two copies), duplication (more than two copies), and reference gap (present as a state to distinguish gaps from homozygous deletions). For example, for a haploid chromosome, there may be no heterozygous deletion state available. In another instance, for trisomic and/or tetrasomic chromosome(s), the HMM states may have an additional intermediate state, wherein the intermediate state can account for the various CNV possibilities. In another embodiment, the HMM is used to filter the output by examination of measured insert-sizes of reads near a detected feature's breakpoint(s).

Other models or algorithms useful in the methods of the present invention include sequential search methods, genetic algorithms, estimation of distribution algorithms, random forest algorithms, weight vector of support vector machine algorithms, weights of logistic regression algorithms, and the like. Bioinformatics. 2007 Oct. 1; 23 (19): 2507-17 provides an overview of the relative merits of the algorithms or models provided above for the analysis of data. Illustrative algorithms include but are not limited to methods that reduce the number of variables such as principal component analysis algorithms, partial least squares methods, independent component analysis algorithms, methods that handle large numbers of variables directly such as statistical methods, and methods based on machine learning techniques. Statistical methods include penalized logistic regression, prediction analysis of microarrays (PAM), methods based on shrunken centroids, support vector machine analysis, and regularized linear discriminant analysis.

Methods and systems provided herein may further include the use of a feature selection algorithm as provided herein. In some embodiments of the present invention, feature selection is provided by use of the LIMMA software package (Smyth, G. K. (2005). Limma: linear models for microarray data. In: Bioinformatics and Computational Biology Solutions using R and Bioconductor, R. Gentleman, V. Carey, S. Dudoit, R. Irizarry, W. Huber (eds.), Springer, New York, pages 397-420).

In some embodiments of the present invention, a diagonal linear discriminant analysis, k-nearest neighbor algorithm, support vector machine (SVM) algorithm, linear support vector machine, random forest algorithm, or a probabilistic model-based method or a combination thereof is provided for the detection of one or more genomic regions. In some embodiments, identified markers that distinguish samples (e.g., diseased versus normal) or distinguish genomic regions (e.g., copy number variation versus normal) are selected based on statistical significance of the difference in expression levels between classes of interest. In some instances, the statistical significance is adjusted by applying a Benjamini Hochberg or another correction for false discovery rate (FDR).

In some instances, the algorithm may be supplemented with a meta-analysis approach such as that described by Fishel and Kaufman et al. 2007 Bioinformatics 23(13): 1599-606. In some instances, the algorithm may be supplemented with a meta-analysis approach such as a repeatability analysis. In some instances, the repeatability analysis selects markers that appear in at least one predictive expression product marker set.

A statistical evaluation of the detection of the genomic regions may provide a quantitative value or values indicative of one or more of the following: the likelihood of diagnostic accuracy; the likelihood of disorder, disease, condition and the like; the likelihood of a particular disorder, disease or condition; and the likelihood of the success of a particular therapeutic intervention. Thus, a physician, who is not likely to be trained in genetics or molecular biology, need not understand the raw data. Rather, the data is presented directly to the physician in the form of the quantitative values or qualitative values to guide patient care. The results can be statistically evaluated using a number of methods known to the art including, but not limited to: the student's T test, the two-sided T test, Pearson rank sum analysis, Hidden Markov Model Analysis, analysis of q-q plots, principal component analysis, one-way ANOVA, two-way ANOVA, LIMMA, and the like.

### XV. Computing Environment

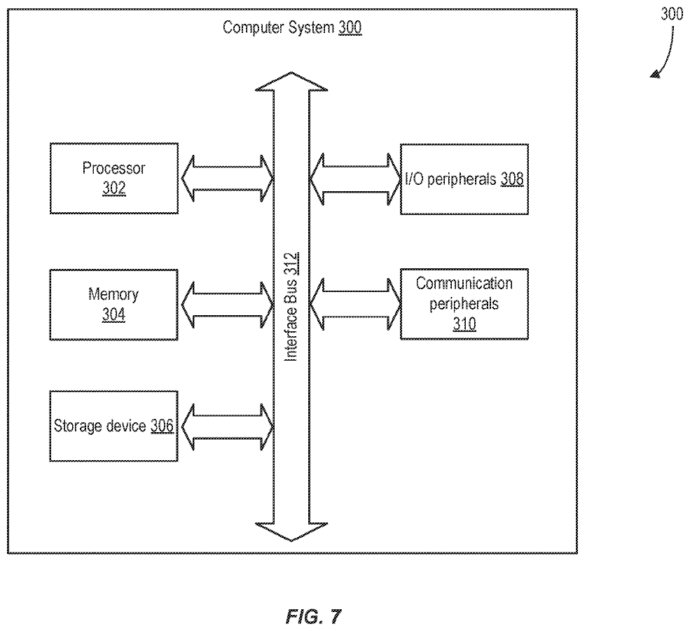

FIG. 7 illustrates an example of a computer system 300 for implementing some of some embodiments disclosed herein. The computer system 300 may include a distributed architecture, where some of the components (e.g., memory and processor) are part of an end user device and some other similar components (e.g., memory and processor) are part of a computer server. In some instances, the computer system 300 is a computer system that for determining a probe-set identifier of a probe set, which includes at least a processor 302, a memory 304, a storage device 306, input/output (I/O) peripherals 308, communication peripherals 310, and an interface bus 312. The interface bus 312 is configured to communicate, transmit, and transfer data, controls, and commands among the various components of computer system 300. The processor 302 may include one or more processing units, such as CPUs, GPUs, TPUs, systolic arrays, or SIMD processors. Memory 304 and storage device 306 include computer-readable storage media, such as RAM, ROM, electrically erasable programmable read-only memory (EEPROM), hard drives, CD-ROMs, optical storage devices, magnetic storage devices, electronic non-volatile computer storage, for example, Flash® memory, and other tangible storage media. Any of such computer-readable storage media can be configured to store instructions or program codes embodying aspects of the disclosure. Memory 304 and storage device 306 also include computer-readable signal media.

A computer-readable signal medium includes a propagated data signal with computer-readable program code embodied therein. Such a propagated signal takes any of a variety of forms including, but not limited to, electromagnetic, optical, or any combination thereof. A computer-readable signal medium includes any computer-readable medium that is not a computer-readable storage medium and that can communicate, propagate, or transport a program for use in connection with computer system 300.

Further, the memory 304 includes an operating system, programs, and applications. The processor 302 is configured to execute the stored instructions and includes, for example, a logical processing unit, a microprocessor, a digital signal processor, and other processors. For example, the computing system 300 can execute instructions (e.g., program code) that configure the processor 302 to perform one or more of the operations described herein. The program code includes, for example, code implementing the analyzing the sequence data, and/or any other suitable applications that perform one or more operations described herein. The instructions could include processor-specific instructions generated by a compiler or an interpreter from code written in any suitable computer-programming language, including, for example, C, C++, C #, Visual Basic, Java, Python, Perl, JavaScript, and ActionScript.

The program code can be stored in the memory 304 or any suitable computer-readable medium and can be executed by the processor 302 or any other suitable processor. In some embodiments, all modules in the computer system for predicting loss of heterozygosity in HLA alleles are stored in the memory 304. In additional or alternative embodiments, one or more of these modules from the above computer system are stored in different memory devices of different computing systems.

The memory 304 and/or the processor 302 can be virtualized and can be hosted within another computing system of, for example, a cloud network or a data center. I/O peripherals 308 include user interfaces, such as a keyboard, screen (e.g., a touch screen), microphone, speaker, other input/output devices, and computing components, such as graphical processing units, serial ports, parallel ports, universal serial buses, and other input/output peripherals. The I/O peripherals 308 are connected to the processor 302 through any of the ports coupled to the interface bus 312. The communication peripherals 310 are configured to facilitate communication between the computer system 300 and other computing devices over a communications network and include, for example, a network interface controller, modem, wireless and wired interface cards, antenna, and other communication peripherals. For example, the computing system 300 is able to communicate with one or more other computing devices (e.g., a computing device that is used for analyzing the sequence data, a computing device that displays outputs the result that includes the probe-set identifier) via a data network using a network interface device of the communication peripherals 310.

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, it should be understood that the present disclosure has been presented for purposes of example rather than limitation, and does not preclude inclusion of such modifications, variations, and/or additions to the present subject matter as would be readily apparent to one of ordinary skill in the art. Indeed, the methods and systems described herein may be embodied in a variety of other forms; furthermore, various omissions, substitutions and changes in the form of the methods and systems described herein may be made without departing from the spirit of the present disclosure. The accompanying claims and their equivalents are intended to cover such forms or modifications as would fall within the scope and spirit of the present disclosure.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provide a result conditioned on one or more inputs. Suitable computing devices include multipurpose microprocessor-based computing systems accessing stored software that programs or configures the computing system from a general-purpose computing apparatus to a specialized computing apparatus implementing one or more embodiments of the present subject matter. Any suitable programming, scripting, or other type of language or combinations of languages may be used to implement the teachings contained herein in software to be used in programming or configuring a computing device.

Certain embodiments of the methods disclosed herein may be performed in the operation of such computing devices. The order of the blocks presented in the examples above can be varied—for example, blocks can be re-ordered, combined, and/or broken into sub-blocks. Certain blocks or processes can be performed in parallel.

Conditional language used herein, such as, among others, “can,” “could,” “might,” “may,” “e.g.,” and the like, unless specifically stated otherwise, or otherwise understood within the context as used, is generally intended to convey that certain examples include, while other examples do not include, certain features, elements, and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Similarly, the use of “based at least in part on” is meant to be open and inclusive, in that a process, step, calculation, or other action “based at least in part on” one or more recited conditions or values may, in practice, be based on additional conditions or values beyond those recited. Headings, lists, and numbering included herein are for ease of explanation only and are not meant to be limiting.

The various features and processes described above may be used independently of one another or may be combined in various ways. All possible combinations and sub-combinations are intended to fall within the scope of the present disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed examples. Similarly, the example systems and components described herein may be configured differently than described. For example, elements may be added to, removed from, or rearranged compared to the disclosed examples.

### EXAMPLES

### A. Example 1

Two types of sequencing are considered. This particular Example corresponds to a single exemplary locus. A first “standard” type of sequencing identifies how many of each allele is detected at the locus. In this illustration, both tumor and normal cells have the same distribution of alleles (10% T and 90% C). (See Table 1.) Therefore, results of the sequencing cannot detect whether the sample includes any tumor cells, much less estimate a relative amount of tumor cells to normal cells.

TABLE 1

| Allele Fraction | Tumor | Normal |
|---|---|---|
| T | 10 | 10 |
| C | 90 | 90 |

A second “methylation” type of sequencing identifies the alleles and further detects methylation. In this illustration, all of the tumor cells' cytosines at the locus are methylated, whereas only 11% (10 divided by 80) of the normal cells' cytosines at the locus are methylated. (See Table 2.) Therefore, analyzing a distribution that distinguishes, not only between alleles, but also between methylated cytosines from unmethylated cytosines, can provide information about the fraction of the cells that are tumor cells.

TABLE 2

| Allele Fraction | Tumor | Normal |
|---|---|---|
| ^{m}C | 90 | 10 |
| T | 10 | 10 |
| C | 0 | 80 |

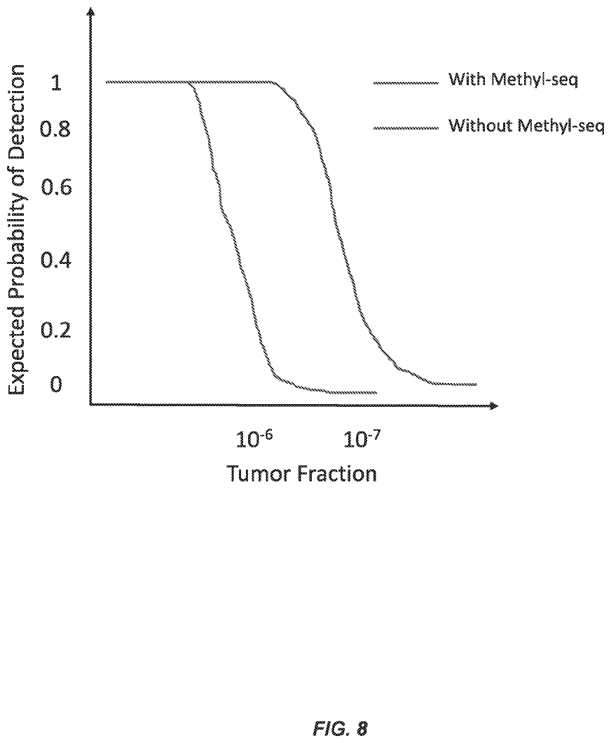

If the fraction of cells is extremely small, the likelihood of being able to detect that there are tumor cells in a given sample may positively correlate with the fraction of cells in the sample that are tumor cells (a “tumor fraction”). For example, if only a single cell in a sample is a tumor cell, it may be impossible or very statistically difficult to detect that it is a tumor cell (and not just noise). FIG. 8 shows a plot of an expected probability of detection versus tumor fraction—both when methylation is considered in addition to bases (so as to indicate any single nucleotide polymorphisms) and when only bases are considered. As shown, the tumor fraction corresponding to a given detection probability is lower when methylation data is available than when it is not. For example, when methylation data is not considered, there is about a 50% detection probability when the tumor fraction is 10-6, whereas the 50% detection probability corresponds to a tumor fraction of about 10-7 when methylation data is considered.

### B. Example 2

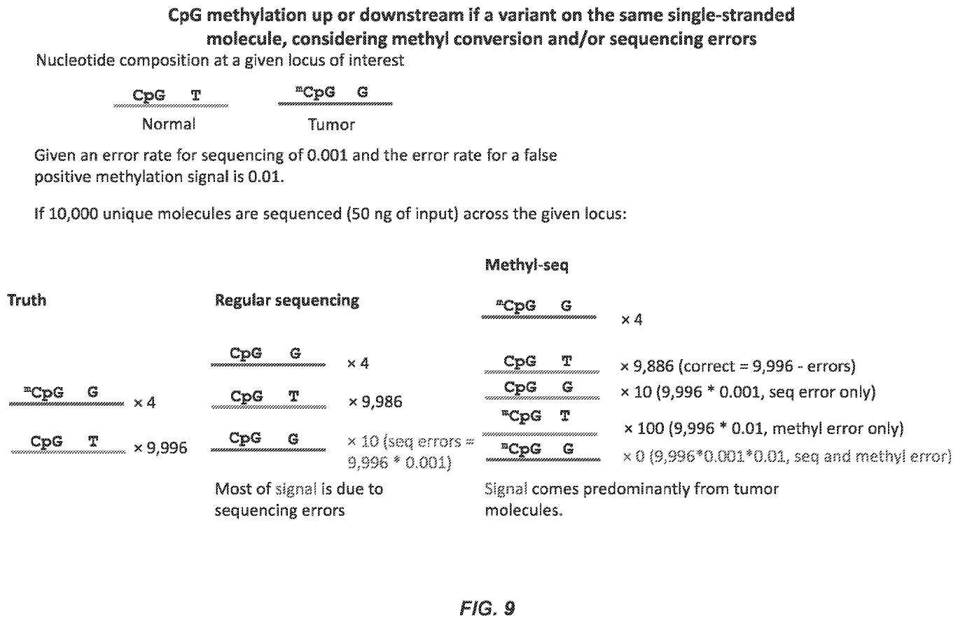

FIG. 9 illustrates a circumstance where a normal sequence includes normal cells with an unmethylated CpG site and a thymine and tumor cells with a methylated CpG site and a guanine. Thus, each of the thymine/guanine base identity and the methylation of the CpG site can be informative as to whether a given read corresponds to a tumor cell or a normal cell.

In this Example, an error rate for sequencing is 0.001, an error rate for a false positive methylation signal is 0.01, and 10,000 unique molecules are sequenced. Additionally, in this Example, the ground-truth for the reads that are sequenced indicate that there are 4 tumor reads and 9,996 normal reads.

When regular sequencing is used, the results indicate that there are 14 CpG G (guanine) reads and 9,986 CpG T (thymine) reads. Of the 14 CpG G reads, 10 are due to sequencing error and 4 are due to the presence of tumor cells. Thus, the majority of the CpG G reads are due to sequencing errors, not tumor cells.

When methylation sequencing is used, the results indicate that there are 4 methylated-CpG G reads due to tumor (true signal), 9,886 unmethylated-CpG T reads due to normal (true signal), 10 unmethylated-CpG G reads (erroneous unmethylation signal), 100 methylated CpG T reads (erroneous methylation signal), and 0 methylated-CpG G reads. Thus, the majority (all) of true methylated-CpG G signal reads are due to the presence of the tumor cells, not sequencing errors. Using both nucleotide and methylation data can more accurately differentiate between true methylated variants and sequencing errors (which will not be methylated). In this example, the probability of 4 methylated CpG G reads occurring due to error is sufficiently low that that just these 4 reads can support a tumor-derived signal call.

### C. Example 3

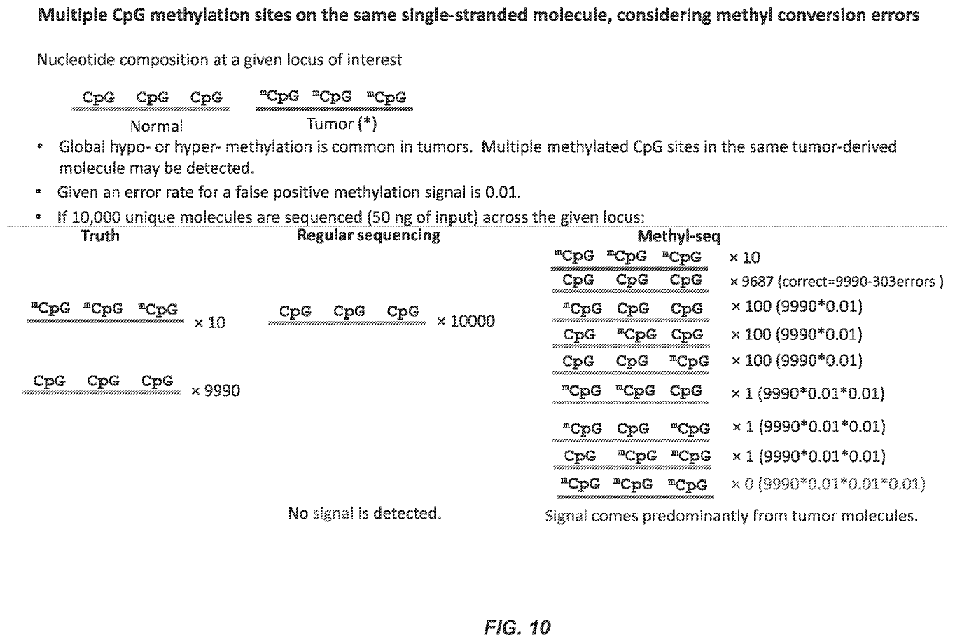

FIG. 10 illustrates a circumstance where a normal sequence includes normal cells with multiple unmethylated CpG sites and tumor cells with multiple methylated CpG sites. Global hypo-or hyper-methylation is common in tumors, and multiple CpG sites in the same tumor derived molecule are detected.

As in Example 2, in this Example, an error rate for a false positive methylation signal is 0.01 and 10,000 unique molecules are sequenced. Additionally, in this Example, the ground-truth for the reads that are sequenced indicate that there are 10 reads with three methylated CpG sites and 9,990 reads with three unmethylated CpG sites.

When regular sequencing is used, the results indicate that all 10,000 of the reads include three unmethylated CpG sites. Thus, no tumor signal is detected.

When methylation sequencing is used, only 96.87% (9687 of 10,000) reads include the three unmethylated CpG sites. The remaining 313 reads include one or more methylated CpG sites. Thus, the majority (all) of reads with three methylated CpG sites are due to the presence of the tumor cells, not sequencing errors.

1-30. (canceled)

31. A method comprising:
(a) accessing sequencing data that had been generated by sequencing a processed sample obtained from a subject, the sequencing data including or having been based on a set of sequence reads;
(b) identifying, using the sequencing data, a plurality of loci corresponding to single nucleotide polymorphisms (SNPs) at which at least a threshold number or percentage of the set of sequence reads included a base identifier that departed from a reference base identifier corresponding to a same position in a reference sequence;
(c) for each locus of the plurality of loci:
(i) determining, for each of one or more positions within a sequence portion that includes the locus, a methylation percentage using reads that include the corresponding SNP; and
(ii) identifying, for each of the one or more positions corresponding to the sequence portion that includes the locus, a comparative methylation percentage, wherein:
(1) a first subset of the plurality of loci is identified using a look-up technique that uses at least part of population-level sequencing data as a first reference sequence; and
(2) a second subset of the plurality of loci is identified using a look-up technique that uses at least part of subject-specific normal sequencing data as a second reference sequence;
(d) generating a result based on each determined methylation percentage and each comparative methylation percentage, wherein the result represents a prediction as to whether the sample is associated with a particular medical condition, whether the sample is associated with a medical condition of a particular stage, whether the subject has a particular type of medical condition, or whether the sample was collected from a specific individual; and
(e) outputting the result.

32. The method of claim 31, wherein the population-level sequencing data is based on or extracted from one or more databases.

33. The method of claim 32, wherein the one or more databases comprises one or more methylation databases or one or more polymorphism databases.

34. The method of claim 32, wherein the one or more databases comprises one or more publicly available databases or one or more proprietary databases.

35. The method of claim 31, wherein the accessed sequencing data was enriched using a plurality of capture probes.

36. The method of claim 35, wherein the plurality of capture probes comprises one or more self-identifying capture probes.

37. The method of claim 35, wherein the plurality of capture probes comprises 1200 or more capture probes.

38. The method of claim 37, wherein the plurality of capture probes comprises 1800 or more capture probes.

39. The method of claim 31, wherein generating the result includes performing a statistical analysis that indicates, for at least one locus of the plurality of loci, a probability of sequencing error accounting for a subset of reads that include the SNP also having the methylation percentage.

40. The method of claim 31, further comprising, for each locus of the plurality of loci:
(i) defining a first subset of reads aligned to at least part of the sequence portion to include reads that include the SNP;
(ii) defining a second subset of reads aligned to at least part of the sequence portion to include reads that do not include the SNP and instead include the reference base identifier; and
(iii) generating, for each position of the one or more positions, the comparative methylation percentage using the methylation state of each cytosine aligned to the position in the second subset of reads.

41. The method of claim 31, further comprising, for a particular locus of the plurality of loci:
(i) detecting, using the sequencing data, one or more CpG sites that are within a predefined number of positions from the SNP; and
(ii) defining the one or more positions to be the loci of the cytosine nucleotide within each of the one or more CpG sites.

42. The method of claim 41, wherein:
(i) the sample was a blood sample;
(ii) the result represents a prediction that the sample is associated with the particular condition; and
(iii) the particular condition includes cancer.

43. The method of claim 42, wherein levels of circulating tumor DNA were below 5 parts per million in the blood sample.

44-48. (canceled)

49. A method comprising:
(a) accessing sequencing data that had been generated by sequencing a processed sample obtained from a subject, the sequencing data including or having been based on a set of sequence reads;
(b) identifying, using the sequencing data, one or more loci corresponding to single nucleotide polymorphisms (SNPs) at which at least a threshold number or percentage of the set of sequence reads included a base identifier that departed from a reference base identifier corresponding to a same position in a reference sequence;
(c) for each locus of the one or more loci:
(i) determining, for each of one or more positions within a sequence portion that includes the locus, a methylation percentage using reads that include the corresponding SNP; and
(ii) identifying, for each of the one or more positions corresponding to the sequence portion that includes the locus, a comparative methylation percentage;
(d) generating a result based on each determined methylation percentage and each comparative methylation percentage, wherein the result represents a prediction as to whether the sample is associated with a particular medical condition, whether the sample is associated with a medical condition of a particular stage, whether the subject has a particular type of medical condition, or whether the sample was collected from a specific individual; and
(e) outputting the result.

50. A method comprising:
(a) accessing solid-tumor sequencing data that had been generated by sequencing a processed sample of a solid tumor obtained from a subject, the sequencing data including or having been based on a set of sequence reads;
(b) determining, for each position of a set of positions in a genome:
(i) a solid-tumor-sample-specific methylation percentage that indicates a first proportion of bases in the solid-tumor sequencing data set that were aligned to the position and were methylated; and
(ii) a comparative methylation percentage that indicates a second proportion of bases in a population sequencing data set or a subject-specific normal sequencing data set, or a combination thereof, that were aligned to the position and were methylated;
(c) determining a subset of the set of positions for which the solid-tumor-sample-specific methylation percentage was sufficiently different from the comparative methylation percentage;
(d) accessing cell-free sequencing data that had been generated by sequencing cell free DNA in a processed or unprocessed sample of the subject;
(e) detecting, for each position of the subset of the set of positions, a quantity of bases aligned to the position that were methylated; and
(f) outputting a result based on, for each position of the subset, the quantity of bases aligned to the position that were methylated.
